(19) United States
(12) Patent Application Publication
(10) Pub. No.: US 2009/0221603 A1
(43) Pub. Date: Sep. 3, 2009

(54) HETEROCYCLIC AMIDE DERIVATIVES AS CALCIUM CHANNEL BLOCKERS

(76) Inventors: Hassan Pajouhesh, West Vancouver (CA); Ramesh Kaul, Burnaby (CA); Yanbing Ding, Richmond (CA); Gabriel Hum, Langley (CA); Hossein Pajouhesh, Coquitlam (CA)

Correspondence Address: MORRISON & FOERSTER LLP, 12531 HIGH BLUFF DRIVE, SUITE 100, SAN DIEGO, CA, 92130-2040, US

(21) Appl. No.: 12/097,035

(22) PCT Filed: Dec. 19, 2006
(86) PCT No.: PCT/CA06/02070

§ 371 Date: Nov. 21, 2008

Related U.S. Application Data
(60) Provisional application No. 60/751,769, filed on Dec. 19, 2005.

(51) Int. Cl. A61K 31/496; A61K 31/495; C07D 241/04; C07D 401/06; A61P 25/00

(52) U.S. Cl. 514/253.01; 514/255.01; 544/391; 544/360

(57) ABSTRACT

Methods and compounds effective in ameliorating conditions characterized by unwanted calcium channel activity, particularly unwanted N-type or T-type calcium channel activity are disclosed. Specifically, a series of heterocyclic amides are disclosed of the general formula (1) where Z is N or ═CHNR₂ and X is NR₂, O, S, S═O or SO₂. Among other definitions for R, R¹, W and Y, the compounds of formula (1) are further characterized by at least one of W or Y being CR³Ar₂ where Ar is an aromatic or heteroaromatic ring (for example, where W or Y is a benzhydryl moiety).

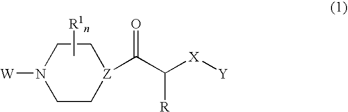

### TECHNICAL FIELD

The invention relates to compounds useful in treating conditions associated with calcium channel function, and particularly conditions associated with N-type and/or T-type calcium channel activity. More specifically, the invention concerns compounds containing substituted or unsubstituted amides adjacent piperazine or piperidine derivatives that are useful in treatment of conditions such as stroke and pain.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Calcium channels mediate a variety of normal physiological functions, and are also implicated in a number of human disorders. Examples of calcium-mediated human disorders include but are not limited to congenital migraine, cerebellar ataxia, angina, epilepsy, hypertension, ischemia, and some arrhythmias. The clinical treatment of some of these disorders has been aided by the development of therapeutic calcium channel antagonists (e.g., dihydropyridines, phenylalkyl amines, and benzothiazapines all target L-type calcium channels) (Janis, R. J. & Triggle, D. J., In Calcium Channels: Their Properties, Functions, Regulation and Clinical Relevance (1991) CRC Press, London).

Native calcium channels have been classified by their electrophysiological and pharmacological properties into T-, L-, N-, P/Q- and R-types (reviewed in Catterall, W., Annu Rev Cell Dev Biol (2000) 16: 521-555; Huguenard, J. R., Annu Rev Physiol (1996) 58: 329-348). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall (2000); Huguenard (1996)). L-type channels can be distinguished by their sensitivity to several classes of small organic molecules used therapeutically, including dihydropyridines (DHP's), phenylalkylamines and benzothiazepines. In contrast, N-type and P/Q-type channels are high affinity targets for certain peptide toxins produced by venomous spiders and marine snails: N-type channels are blocked by the ω-conopeptides ω-conotoxin GVIA (ω-CTx-GVIA) isolated from Conus geographus and ω-conotoxin MVIIA (ω-CTx-MVIIA) isolated from Conus magus, while P/Q-type channels are resistant to ω-CTx-MVIIA but are sensitive to the funnel web spider peptide, ω-agatoxin IVA (ω-Aga-IVA). R-type calcium channels are sensitive to block by the tarantula toxin, SNX-482.

Neuronal high voltage-activated calcium channels are composed of a large (>200 kDa) pore-forming α₁ subunit that is the target of identified pharmacological agents, a cytoplasmically localized ˜50-70 kDa β subunit that tightly binds the α₁ subunit and modulates channel biophysical properties, and an ˜170 kDa α₂δ subunit (reviewed by Stea, et al., Proc Natl Acad Sci USA (1994) 91:10576-10580; Catterall (2000)). At the molecular level, nine different α₁ subunit genes expressed in the nervous system have been identified and shown to encode all of the major classes of native calcium currents (Table 1).

TABLE 1
Classification of Neuronal Calcium Channels

| | | | ω- | | | |
|---|---|---|---|---|---|---|
| Native | | Gene | AGA | ω-CTx | ω-CTx | |
| Class | cDNA | Name | IVA | GVIA | MVIA | dihydropyridines |
| P/Q- | α_{1A} | Ca_{V}2.1 | ✓ | — | — | — |
| type | | | | | | |
| N-type | α_{1B} | Ca_{V}2.2 | — | ✓ | ✓ | — |
| L-type | α_{1C} | Ca_{V}1.2 | — | — | — | ✓ |
| L-type | α_{1D} | Ca_{V}1.3 | — | — | — | ✓ |
| R-type | α_{1E} | Ca_{V}2.3 | — | — | — | — |
| L-type | α_{1F} | Ca_{V}1.4 | — | — | — | ✓ |
| T-type | α_{1G} | Ca_{V}3.1 | — | — | — | — |
| T-type | α_{1H} | Ca_{V}3.2 | — | — | — | — |
| T-type | α_{1I} | Ca_{V}3.3 | — | — | — | — |

Calcium channels have been shown to mediate the development and maintenance of the neuronal sensitization processes associated with neuropathic pain, and provide attractive targets for the development of analgesic drugs (reviewed in Vanegas, H. & Schaible, H-G., Pain (2000) 85: 9-18). All of the high-threshold Ca channel types are expressed in the spinal cord, and the contributions of L-, N and P/Q-types in acute nociception are currently being investigated. In contrast, examination of the functional roles of these channels in more chronic pain conditions strongly indicates a pathophysiological role for the N-type channel (reviewed in Vanegas & Schaible (2000) supra).

Mutations in calcium channel α₁ subunit genes in animals can provide important clues to potential therapeutic targets for pain intervention. Genetically altered mice null for the α_{1B} N-type calcium channel gene have been reported by several independent groups (Ino, M. et al., Proc Natl Acad Sci USA (2001) 98(9): 5323-5328; Kim, C. et al., Mol Cell Neurosci (2001) 18(2): 235-245; Saegusa, H. et al., Proc Natl Acad Sci USA (2001) 97: 6132-6137; Hatakeyama, S. et al., Neuroreport (2001) 12(11): 2423-2427). The α_{1B} N-type null mice were viable, fertile and showed normal motor coordination. In one study, peripheral body temperature, blood pressure and heart rate in the N-type gene knock-out mice were all normal (Saegusa, et al. (2001)). In another study, the baroreflex mediated by the sympathetic nervous system was reduced after bilateral carotid occlusion (Ino, et al. (2001)). In another study, mice were examined for other behavioral changes and were found to be normal except for exhibiting significantly lower anxiety-related behaviors (Saegusa, et al. (2001)), suggesting the N-type channel may be a potential target for mood disorders as well as pain. In all studies, mice lacking functional N-type channels exhibit marked decreases in the chronic and inflammatory pain responses. In contrast, mice lacking N-type channels generally showed normal acute nociceptive responses.

Two examples of either FDA-approved or investigational drug that act on N-type channel are gabapentin and ziconotide. Gabapentin, 1-(aminomethyl)cyclohexaneacetic acid (Neurontin®), is an anticonvulsant originally found to be active in a number of animal seizure models (Taylor, C. P. et al., Epilepsy Res (1998) 29: 233-249). Subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanoallodynia associated with postoperative pain (Taylor, et al. (1998); Cesena, R. M. & Calcutt, N. A., Neurosci Lett (1999) 262: 101-104; Field, M. J. et al., Pain (1999) 80: 391-398; Cheng, J-K., et al., Anesthesiology (2000) 92: 1126-1131; Nicholson, B., Acta Neurol Scand (2000) 101: 359-371).

While its mechanism of action is not completely understood, current evidence suggests that gabapentin does not directly interact with GABA receptors in many neuronal systems, but rather modulates the activity of high threshold calcium channels. Gabapentin has been shown to bind to the calcium channel α₂δ ancillary subunit, although it remains to be determined whether this interaction accounts for its therapeutic effects in neuropathic pain.

In humans, gabapentin exhibits clinically effective anti-hyperalgesic activity against a wide ranging of neuropathic pain conditions. Numerous open label case studies and three large double blind trials suggest gabapentin might be useful in the treatment of pain. Doses ranging from 300-2400 mg/day were studied in treating diabetic neuropathy (Backonja, M. et al., JAMA (1998) 280:1831-1836), postherpetic neuralgia (Rowbotham, M. et al., JAMA (1998) 280: 1837-1842), trigeminal neuralgia, migraine and pain associated with cancer and multiple sclerosis (Di Trapini, G. et al., Clin Ter (2000) 151: 145-148; Caraceni, A. et al., J Pain & Symp Manag (1999) 17: 441-445; Houtchens, M. K. et al., Multiple Sclerosis (1997) 3: 250-253; see also Magnus, L., Epilepsia (1999) 40(Suppl 6): S66-S72; Laird, M. A. & Gidal, B. E., Annal Pharmacotherap (2000) 34: 802-807; Nicholson, B., Acta Neurol Scand (2000) 101: 359-371).

Ziconotide (Prialt®; SNX-111) is a synthetic analgesic derived from the cone snail peptide Conus magus MVIIA that has been shown to reversibly block N-type calcium channels. In a variety of animal models, the selective block of N-type channels via intrathecal administration of Ziconotide significantly depresses the formalin phase 2 response, thermal hyperalgesia, mechanical allodynia and post-surgical pain (Malmberg, A. B. & Yaksh, T. L., J Neurosci (1994) 14: 4882-4890; Bowersox, S. S. et al., J Pharmacol Exp Ther (1996) 279: 1243-1249; Sluka, K. A., J Pharmacol Exp Ther (1998) 287:232-237; Wang, Y-X. et al., Soc Neurosci Abstr (1998) 24: 1626).

Ziconotide has been evaluated in a number of clinical trials via intrathecal administration for the treatment of a variety of conditions including post-herpetic neuralgia, phantom limb syndrome, HIV-related neuropathic pain and intractable cancer pain (reviewed in Mathur, V. S., Seminars in Anesthesia, Perioperative medicine and Pain (2000) 19: 67-75). In phase II and III clinical trials with patients unresponsive to intrathecal opiates, Ziconotide has significantly reduced pain scores and in a number of specific instances resulted in relief after many years of continuous pain. Ziconotide is also being examined for the management of severe post-operative pain as well as for brain damage following stroke and severe head trauma (Heading, C., Curr Opin CPNS Investigational Drugs (1999) 1: 153-166). In two case studies Ziconotide has been further examined for usefulness in the management of intractable spasticity following spinal cord injury in patients unresponsive to baclofen and morphine (Ridgeway, B. et al., Pain (2000) 85: 287-289). In one instance Ziconotide decreased the spasticity from the severe range to the mild to none range with few side effects. In another patient Ziconotide also reduced spasticity to the mild range although at the required dosage significant side effects including memory loss, confusion and sedation prevented continuation of the therapy.

T-type calcium channels are involved in various medical conditions. In mice lacking the gene expressing the α_{1G} subunit, resistance to absence seizures was observed (Kim, C. et al., Mol Cell Neurosci (2001) 18(2): 235-245). Other studies have also implicated the α_{1H} subunit in the development of epilepsy (Su, H. et al., J Neurosci (2002) 22: 3645-3655). There is strong evidence that some existing anticonvulsant drugs, such as ethosuximide, function through the blockade of T-type channels (Gomora, J. C. et al., Mol Pharmacol (2001) 60: 1121-1132).

Low voltage-activated calcium channels are highly expressed in tissues of the cardiovascular system. Mibefradil, a calcium channel blocker 10-30-fold selective for T-type over L-type channels, was approved for use in hypertension and angina. It was withdrawn from the market shortly after launch due to interactions with other drugs (Heady, T. N., et al., Jpn J. Pharmacol. (2001) 85:339-350).

Growing evidence suggests T-type calcium channels may also be involved in pain. Both mibefradil and ethosuximide have shown anti-hyperalgesic activity in the spinal nerve ligation model of neuropathic pain in rats (Dogrul, A., et al., Pain (2003) 105:159-168).

U.S. Pat. Nos. 6,011,035; 6,294,533; 6,310,059; and 6,492,375; PCT publications WO 01375 and WO 01/45709; PCT publications based on PCT CA 99/00612, PCT CA 00/01586; PCT CA 00/01558; PCT CA 00/01557; PCT CA 2004/000535; and PCT CA 2004/000539, and U.S. patent application Ser. Nos. 10/746,932 filed 23 Dec. 2003; 10/746,933 filed 23 Dec. 2003; 10/409,793 filed 8 Apr. 2003; 10/409,868 filed 8 Apr. 2003; 10/655,393 filed 3 Sep. 2003; 10/821,584 filed 9 Apr. 2004; and 10/821,389 filed 9 Apr. 2004 disclose calcium channel blockers where a piperidine or piperazine ring is substituted by various aromatic moieties. These applications and publications are incorporated herein by reference.

U.S. Pat. No. 5,646,149 describes calcium channel antagonists of the formula A-Y—B wherein B contains a piperazine or piperidine ring directly linked to Y. An essential component of these molecules is represented by A, which must be an antioxidant; the piperazine or piperidine itself is said to be important. The exemplified compounds contain a benzhydryl substituent, based on known calcium channel blockers (see below). U.S. Pat. No. 5,703,071 discloses compounds said to be useful in treating ischemic diseases. A mandatory portion of the molecule is a tropolone residue, with substituents such as piperazine derivatives, including their benzhydryl derivatives. U.S. Pat. No. 5,428,038 discloses compounds indicated to exhibit a neural protective and antiallergic effect. These compounds are coumarin derivatives which may include derivatives of piperazine and other six-membered heterocycles. A permitted substituent on the heterocycle is diphenylhydroxymethyl. U.S. Pat. No. 6,458,781 describes 79 amides as calcium channel antagonists though only a couple of which contain both piperazine rings and benzhydryl moieties. Thus, approaches in the art for various indications which may involve calcium channel blocking activity have employed compounds which incidentally contain piperidine or piperazine moieties substituted with benzhydryl but mandate additional substituents to maintain functionality.

Certain compounds containing both benzhydryl moieties and piperidine or piperazine are known to be calcium channel antagonists and neuroleptic drugs. For example, Gould, R. J., et al., Proc Natl Acad Sci USA (1983) 80:5122-5125 describes antischizophrenic neuroleptic drugs such as lidoflazine, fluspirilene, pimozide, clopimozide, and penfluridol. It has also been shown that fluspirilene binds to sites on L-type calcium channels (King, V. K, et al., J Biol Chem (1989) 264:5633-5641) as well as blocking N-type calcium current (Grantham, C. J., et al., Brit J Pharmacol (1944) 111:483-488). In addition, Lomerizine, as developed by Kanebo, K. K., is a known calcium channel blocker. However, Lomerizine is not specific for N-type channels. A review of publications concerning Lomerizine is found in Dooley, D., Current Opinion in CPNS Investigational Drugs (1999) 1:116-125.

All patents, patent applications and publications identified herein are herein incorporated by reference in their entirety.

### DISCLOSURE OF THE INVENTION

The invention relates to compounds useful in treating conditions modulated by calcium channel activity and in particular conditions mediated by N-type and/or T-type calcium channel activity. The compounds of the invention are amides of heterocyclic rings, specifically piperazine or piperidine rings with substituents that enhance the calcium channel blocking activity of the compounds. Thus, in one aspect, the invention is directed to a method of treating conditions mediated by calcium channel activity by administering to patients in need of treatment compounds of the formula

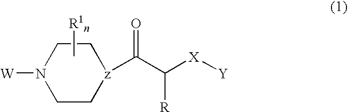

and pharmaceutically acceptable salts or conjugates thereof

wherein X is NR₂, O, S, S═O, or SO₂;

Z is N or CHNR₂;

Y is CR³Ar₂ or Ar;

W is CR³Ar₂, CR³AB or C═OA,

wherein at least one of W and Y is CR³Ar₂;

each Ar is independently an optionally substituted aromatic or heteroaromatic ring;

A is an optionally substituted aromatic or heteroaromatic ring, or an optionally substituted carbocyclic or heterocyclic ring;

B is halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or B may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-1° C.), heteroaryl (5-12C), O-aryl (6-1° C.), O-heteroaryl (5-12C) and C6-C12-aryl-C1-C8-alkyl;

R is H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or R may be an optionally substituted group selected from alkyl (1-8C), alkenyl (2-8C), or alkynyl (2-8C), heteroalkyl (2-8C), heteroalkenyl (2-8C), heteroalkynyl (2-8C), aryl (6-10C), heteroaryl (5-12C), O-aryl (6-10C), O-heteroaryl (5-12C) and C6-C12-aryl-C1-C8-alkyl;

each R¹ is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R¹ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-1° C.), heteroaryl (5-12C), O-aryl (6-10C), O-heteroaryl (5-12C) and C6-C12-aryl-C1-C8-alkyl;

each R² is independently H, or an optionally substituted group selected from alkyl (1-8C), alkenyl (2-8C) and alkynyl (2-8C);

each R³ is independently H, halo, CN, OR′, SR′, SOR′, SO₂R′, NR¹², NR′(CO)R′, or NR′SO₂R′, wherein each R′ is independently H or an optionally substituted group selected from alkyl (1-6C), heteroaryl (5-12C), and aryl (6-10); or R³ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), and C6-C12-aryl-C1-C8-alkyl;

n is 0-4; and

one or more optional substituents may be on one or more of Ar, A or B wherein, when the substituents on Ar, A or B is on an aromatic or heteroaromatic group, each optional substituent is independently selected from halo, CN, NO₂, CF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10C); or each optional substituent may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10C), heteroaryl (5-12C), O-aryl (6-10C), O-heteroaryl (5-12C) and C6-C12-aryl-C1-C8-alkyl; and

when the substituents on A or B is on a non-aromatic group, each substituent is independently selected from the group consisting of ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10C); or each substituent may be independently selected from alkyl (1-8C), alkenyl (2-8C), or alkynyl (2-8C), heteroalkyl (2-8C), heteroalkenyl (2-8C), heteroalkynyl (2-8C), aryl (6-10C), heteroaryl (5-12C), O-aryl (6-1° C.), O-heteroaryl (5-12C) and C6-C12-aryl-C1-C8-alkyl.

The invention is also directed to compounds of formula (1) useful to modulate calcium channel activity, particularly N-type and T-type channel activity, wherein the definition of such compound is as above with the additional provisos that if X is O and Y is phenyl, then Y is unsubstituted and that if W is CR³AB and A is pyridine, then A is unsubstituted. The invention is also directed to the use of these compounds for the preparation of medicaments for the treatment of conditions requiring modulation of calcium channel activity, and in particular N-type calcium channel activity. In another aspect, the invention is directed to pharmaceutical compositions containing the compounds of formula (1).

### DEFINITIONS

As used herein, the term “alkyl,” “alkenyl” and “alkynyl” include straight-chain, branched-chain and cyclic monovalent substituents, as well as combinations of these, containing C and H. Examples include methyl, ethyl, isobutyl, cyclohexyl, cyclopentylethyl, 2-propenyl, 3-butynyl, and the like. Typically, the alkyl, alkenyl and alkynyl groups contain 1-8C (alkyl) or 2-8C (alkenyl or alkynyl). In some embodiments, they contain 1-6C or 1-4C (alkyl); or 2-6C or 2-4C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby one carbon atom is replaced by one of these heteroatoms. In preferred embodiments, the heteroatom is O or N. For greater certainty, to the extent that alkyl is defined as 1-8C, then the corresponding heteroalkyl contains 2-8 C, N, O, or S atoms such that the heteroalkyl contains at least one C atom and at least one heteroatom. Similarly, when alkyl is defined as 1-6C or 1-4C, the heteroform would be 2-6C or 2-4C respectively, wherein one C is replaced by O, N or S. Accordingly, when alkenyl or alkynyl is defined as 2-8C (or 2-6C or 2-4C), then the corresponding heteroform would also contain 2-8 C, N, O, or S atoms (or 2-6 or 2-4 respectively) since the heteroalkenyl or heteroalkynyl contains at least one carbon atom and at least one heteroatom. Further, heteroalkyl, heteroalkenyl or heteroalkynyl substituents may also contain one or more carbonyl groups. Examples of heteroalkyl, heteroalkenyl and heteroalkynyl substituents include CH₂OCH₃, CH₂N(CH₃)₂, CH₂OH, (CH₂)ₙNR₂, OR, COOR, CONR₂, (CH₂)ₙOR, (CH₂), COR, (CH₂)ₙCOOR, (CH₂)ₙCONR₂, NRCOR, NRCOOR, OCONR₂, OCOR and the like wherein the substituent contains at least one C and the size of the substituent is consistent with the definition of alkyl, alkenyl and alkynyl.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Typically, the ring systems contain 5-12 ring member atoms. In some embodiments, the aromatic or heteroaromatic moiety is a 6-membered aromatic rings system optionally containing 1-2 nitrogen atoms. More particularly, the moiety is an optionally substituted phenyl, 2-, 3- or 4-pyridyl, indolyl, 2- or 4-pyrimidyl, pyridazinyl, benzothiazolyl or benzimidazolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C or more particularly 1-6C or 1-4C when saturated or 2-8C, 2-6C or 2-4C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl, and 2-phenylvinyl, commonly referred to as styryl.

“Carbocyclic” moiety refers to any monocyclic or fused ring bicyclic system that is not aromatic and may be unsaturated or saturated but containing only carbon atoms along the backbone; “heterocyclic” refers to any carbocyclic moiety containing one or more heteroatoms selected from O, S and N as ring members. Further, a heterocyclic ring may also contain a carbonyl group wherein the carbon in the carbonyl is a member of the ring. Examples of carbocyclic/heterocyclic rings systems include cyclohexyl, cyclopentyl, cycloheptyl, cyclooctyl, pyrrolidinyl, piperidinyl, morpholinyl, β-lactams, γ-lactones, pyranyl, tetrahydro-2H-pyranyl and the like. Typically, the ring systems contain 5-12 ring member atoms, for example 5-6, and more particularly 6 atoms. In some embodiments, the ring system contains 6 ring member atoms optionally containing 1 nitrogen or oxygen atom. In particular embodiments, the carbocyclic or heterocyclic ring is cyclohexyl, 1-methyl-piperidin-4-yl, or tetrahydro-2H-pyran-4-yl.

When the substituents on Ar, A or B is on an aromatic or heteroaromatic group, typical optional substituents are independently halo, CN, NO₂, CF₃, COOR′, CONR′₂, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or the substituent may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10C), heteroaryl (5-12C), O-aryl (6-10), O-heteroaryl (5-12C) and C6-C12-aryl-C1-C8-alkyl.

When the optional substituents on A or B is on a non-aromatic group, the substituents are typically selected from ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or it may be alkyl (1-8C), alkenyl (2-8C), or alkynyl (2-8C), heteroalkyl (2-8C), heteroalkenyl (2-8C), heteroalkynyl (2-8C), aryl (6-10C), heteroaryl (5-12C), O-aryl (5-10C), O-heteroaryl (5-12C) and C6-C12-aryl-C1-C8-alkyl. For greater certainty, two substituents on the same N or adjacent C can form a 5-7 membered ring which may contain one or two additional heteroatoms selected from N, O and S.

Halo may be any halogen atom, especially F, Cl, Br, or I, and more particularly it is fluoro or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures (the substituents on Ar, A, or B) above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

R may be H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R may be an optionally substituted group selected from alkyl (1-8C), alkenyl (2-8C), or alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl. In particular embodiments, R may be H or 1-8C alkyl, a 1-6C alkyl or even more particularly a 1-4C alkyl. In specific examples, R may be H, methyl, ethyl, isopropyl, propyl, cyclopropyl, n-butyl or isobutyl. In a preferred embodiment, R is H.

There may be from 0-4 substituents (defined as R¹) on the central piperazine or piperidine ring and more particularly 0-2 substituents. Each R¹ may independently be ═O, alkyl, alkenyl, alkynyl, heteroalkyl, heteroalkenyl, heteroalkynyl, aryl, heteroaryl, alkylaryl, O-aryl, O-heteroaryl, halo, CN, OH, NO₂, or NH₂. Where it makes sense chemically, each of these groups (other than H) can be substituted. In more particular embodiments, R¹ may be 1-8C alkyl or heteroalkyl, more particularly a 1-6C alkyl or heteroalkyl or a 1-4C alkyl or heteroalkyl. For example, R¹ may be CH₃, CH₂OH, CH₂OCH₃, CH₂OCH₂COOH, COOH, CH₂OCH₂CH₂OH, CH₂N(CH₃)₂, CH₂—O—(CH₂)₂N(CH₃)₂, COOCH₂CH₂N(CH₃)₂, COO(CH₂)COOH. It may also be ═O, in which case n is typically 1 or 2. In one embodiment, when n equals 2, then R¹ may be 2,6-dimethyl when Z is counted as position 1. In other particular embodiments when n equals 1, R¹ may be methyl, CH₂OH or CH₂OCH₃.

Each R² may independently be H, alkyl, alkenyl or alkynyl, for example. Where it makes sense chemically, each of these groups (other than H) can be substituted. In more particular embodiments, R² is H or 1-8 C alkyl, more particularly 1-6 C alkyl or 1-4 C alkyl. In even more particular embodiments R² is H or methyl.

Each R³ may independently be H, alkyl, alkenyl, alkynyl, heteroalkyl, heteroalkenyl, heteroalkynyl, halo, CN, OH, NO₂, or NH₂, for example. Where it makes sense chemically, each of these groups (other than H) can be substituted. In more particular embodiments, R³ may be H, 1-4C alkyl, CN or OH. In an even more particular embodiment R³ is H.

Each Ar is independently an optionally substituted aromatic or heteroaromatic ring as defined above. “A” encompasses the definition of Ar but may also be a carbocyclic or heterocyclic ring. “B” is defined to include additional groups such as H, alkyl, alkenyl, or alkynyl, heteroalkyl, heteroalkenyl, heteroalkynyl, aryl, heteroaryl, alkylaryl, O-aryl, O-heteroaryl, halo, CN, OH, NO₂, or NH₂. Where it makes sense chemically, each of these groups (other than H or halo) can be substituted. Since the definition of alkyl, alkenyl, alkynyl and their respective heteroforms includes cyclic moieties, the definition of “B” substantially encompasses the definition of “A” except that the size of a carbocyclic ring is potentially larger than allowed by the definition of alkyl.

“Y” is defined as either CR³Ar₂ or simply Ar. “W” is defined as CR³Ar₂, CR³AB or C═OA. It can be noted that the definition of W being equal to CR³AB is broad enough to encompass W also being equal to CR³Ar₂ since both A and B can be aromatic or heteroaromatic rings (i.e. Ar). Nevertheless, for the sake of clarity, the definition of W explicitly includes CR³Ar₂ since the scope of the present compounds requires that at least one of Y and W be equal to CR³Ar₂. In a particular embodiment, R³ is H and both Ar are phenyl, and accordingly CR³Ar₂ is a benzhydryl moiety. Thus in a particular embodiment, at least one of Y and W is a benzhydryl moiety. Optionally, this benzhydryl group may be substituted at the methine carbon or on one or both phenyl rings.

The central ring may be either a piperazine ring when Z is N or a piperidine ring when Z is CHNR₂ (where R² is as defined above). In a more particular embodiment, the central ring is a piperazine ring. The nitrogen atom from Z is coupled to a carbonyl to form an amide and spaced two atoms from this amide is a heteroatom which can be N, O or S. Accordingly, X may be NR₂, O, S, S═O or SO₂ and more particularly, X may be NH, NCH₃, O, S or S═O.

In some preferred embodiments, two or more of the particularly described groups are combined into one compound: it is often suitable to combine one of the specified embodiments of one feature as described above with a specified embodiment or embodiments of one or more other features as described above. For example, a specified embodiment includes R═H, and another specified embodiment has R²═H or methyl. Thus one preferred embodiment combines both of these features together, i.e., R═H in combination with R²═H or R═H in combination with R²=methyl.

Another specified embodiment includes R³ as H; thus one preferred embodiment has R³═H in combination with R═H; another has R³═H in combination with R²═H or Me; and a third has R³═H in combination with R═H and R²═H or Me.

Other specified embodiments have a benzhydryl group for at least one of W and Y; thus preferred embodiments include W=benzyhdryl in combination with any of the preferred combinations set forth above, or Y=benzyhdryl in combination with any of the preferred combinations set forth above.

Other specified embodiments have Z=N. Thus additional preferred embodiments include Z=N in combination with any of the preferred combinations set forth above.

In some specific embodiments, n is 0 to 2. Thus additional preferred embodiments include n=0 in combination with any of the preferred combinations set forth above; other preferred combinations include n=1 in combination with any of the preferred combinations set forth above; and other preferred combinations include n=2 in combination with any of the preferred combinations set forth above.

In some specific embodiments, X is NH or NMe; thus in some preferred combinations, X═NH in combination with any of the preferred combinations set forth above. In other preferred combinations, X═NMe in combination with any of the preferred combinations set forth above.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures.

Compounds of formula (1) are also useful for the manufacture of a medicament useful to treat conditions characterized by undesired N-type and/or T-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

### MODES OF CARRYING OUT THE INVENTION

The compounds of formula (1) including compounds where the provisos do not apply are useful in the methods of the invention and exert their desirable effects through their ability to modulate the activity of N-type and/or T-type calcium channels. The compounds of formula (1) are particularly useful in modulating the activity of N-type calcium channels. This makes them useful for treatment of certain conditions. Conditions where modulation of N-type calcium channels is desired include: chronic and acute pain; mood disorders such as anxiety, depression, and addiction; neurodegenerative disorders; gastrointestinal disorders such as inflammatory bowel disease and irritable bowel syndrome; genitourinary disorders such as urinary incontinence, interstitial colitis and sexual dysfunction; neuroprotection such as cerebral ischemia, stroke and traumatic brain injury; and metabolic disorders such as diabetes and obesity. Conditions where modulation of T-type calcium channels is desired include: cardiovascular disease; epilepsy; diabetes; certain types of cancer such as prostate cancer; chronic and acute pain; sleep disorders; Parkinson's disease; psychosis such as schizophrenia; and male birth control.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

Anxiety as used herein includes but is not limited to the following conditions: generalized anxiety disorder, social anxiety disorder, panic disorder, obsessive-compulsive disorder, and post-traumatic stress syndrome. Addiction includes but is not limited to dependence, withdrawal and/or relapse of cocaine, opioid, alcohol and nicotine.

Neurodegenerative disorders as used herein include Parkinson's disease, Alzheimer's disease, multiple sclerosis, neuropathies, Huntington's disease and amyotrophic lateral sclerosis (ALS).

Cardiovascular disease as used herein includes but is not limited to hypertension, pulmonary hypertension, arrhythmia (such as atrial fibrillation and ventricular fibrillation), congestive heart failure, and angina pectoris.

Epilepsy as used herein includes but is not limited to partial seizures such as temporal lobe epilepsy, absence seizures, generalized seizures, and tonic/clonic seizures.

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

While the compounds described above generally have this activity, availability of this class of calcium channel modulators permits a nuanced selection of compounds for particular disorders. The availability of this class of compounds provides not only a genus of general utility in indications that are affected by calcium channel activity, but also provides a large number of compounds which can be mined and manipulated for specific interaction with particular forms of calcium channels. Compounds may be active against both N-type and T-type calcium channels and that may be of particular benefit for certain disorders, particularly those indications modulated by both N-type and T-type calcium channels. However, for some indications, it may be desirable to have a compound that selectively modulates N-type or T-type calcium channels. The availability of recombinantly produced calcium channels of the α_{1A}-α_{1I} and α_{1S} types set forth above, facilitates this selection process. Dubel, S. J., et al., Proc. Natl. Acad. Sci. USA (1992) 89:5058-5062; Fujita, Y., et al., Neuron (1993) 10:585-598; Mikami, A., et al., Nature (1989) 340:230-233; Mori, Y., et al., Nature (1991) 350:398-402; Snutch, T. P., et al., Neuron (1991) 7:45-57; Soong, T. W., et al., Science (1993) 260:1133-1136; Tomlinson, W. J., et al., Neuropharmacology (1993) 32:1117-1126; Williams, M. E., et al., Neuron (1992) 8:71-84; Williams, M. E., et al., Science (1992) 257:389-395; Perez-Reyes, et al., Nature (1998) 391:896-900; Cribbs, L. L., et al., Circulation Research (1998) 83:103-109; Lee, J. H., et al., Journal of Neuroscience (1999) 19:1912-1921; McRory, J. E., et al., Journal of Biological Chemistry (2001) 276:3999-4011.

It is known that calcium channel activity is involved in a multiplicity of disorders, and particular types of channels are associated with particular conditions. The association of N-type and T-type channels in conditions associated with neural transmission would indicate that compounds of the invention which target N-type receptors are most useful in these conditions. Many of the members of the genus of compounds of formula (1) exhibit high affinity for N-type channels and/or T-type channels. Thus, as described below, they are screened for their ability to interact with N-type and/or T-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

There are three distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

A third type of inhibition is designated “resting channel block”. Resting channel block is the inhibition of the channel that occurs in the absence of membrane depolarization, that would normally lead to opening or inactivation. For example, resting channel blockers would diminish the peak current amplitude during the very first depolarization after drug application without additional inhibition during the depolarization.

In order to be maximally useful in treatment, it is also helpful to assess the side reactions which might occur. Thus, in addition to being able to modulate a particular calcium channel, it is desirable that the compound has very low activity with respect to the HERG K⁺ channel which is expressed in the heart. Compounds that block this channel with high potency may cause reactions which are fatal. Thus, for a compound that modulates the calcium channel, it should also be shown that the HERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Examples 19-22.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, i.e., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) may be used alone, as mixtures of two or more compounds of formula (1) or in combination with other pharmaceuticals. An example of other potential pharmaceuticals to combine with the compounds of formula (1) would include pharmaceuticals for the treatment of the same indication but having a different mechanism of action from N-type or T-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.1-15 mg/kg, preferably 0.1-1 mg/kg. However, dosage levels are highly dependent on the nature of the condition, drug efficacy, the condition of the patient, the judgment of the practitioner, and the frequency and mode of administration.

Synthesis of the Invention Compounds

The following examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

### Example 1

### Synthesis of Acetic Acid Intermediates

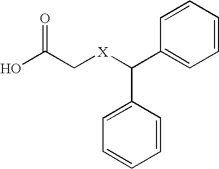

### A. Synthesis of 2-(benzhydryl(methyl)amino)acetic acid

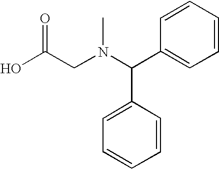

To a solution of N-diphenylmethyl-methylamine 1.97 g (10 mmol) in acetonitrile (20 ml) was added ethyl bromoacetate 1.2 ml (11 mmol) and potassium carbonate 1.38 g (10 mmol). The reaction mixture was refluxed for two hours, concentrated, water was added and the product was then extracted with ethyl acetate 50 ml. The organic solution was dried over sodium sulfate and concentrated to give 3 g of crude ester. To the ester, lithium hydroxide 1.25 g (30 mmol) and methanol (10 ml), THF (30 ml) and water (10 ml) was added. The mixture was stirred at room temperature overnight, concentrated to remove solvent, neutralized with 2N HCl to pH˜3, and extracted with ethyl acetate (40 ml). The organic layer was dried over sodium sulfate and concentrated to give 2.2 g of desired product.

### B. Synthesis of 2-(benzhydrylamino)acetic acid

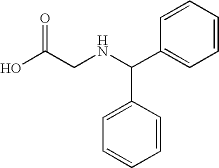

To a solution of aminodiphenylmethane 1.85 g (10 mmol) in DMF (20 ml) was added ethyl bromoacetate 1.2 ml (11 mmol) and potassium carbonate 1.38 g (10 mmol). The reaction mixture was heated at about 60° C. for two days, concentrated, water was added and the product was then extracted with ethyl acetate 2×50 ml. The organic solution was dried over sodium sulfate and concentrated to give 3 g of crude ester. To the ester, lithium hydroxide 1.25 g (30 mmol) and methanol (10 ml), THF (30 ml) and water (10 ml) was added. The mixture was stirred at room temperature overnight, concentrated to remove solvent, neutralized with 2N HCl to pH˜3, and extracted with ethyl acetate 40 ml. The organic layer was dried over sodium sulfate and concentrated to give 2.0 g of desired product.

### C. Synthesis of 2-(benzhydryloxy)acetic acid

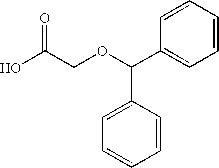

To a solution of benzhydrol 3.68 g (20 mmol) in THF (40 ml) was added sodium, hydride 1 g (24 mmol). The reaction mixture was stirred at room temperature for half an hour. 2.4 ml ethyl bromoacetate (22 mmol) was added, and the reaction mixture was stirred at room temperature overnight. The reaction was quenched with methanol, concentrated, water was added and the product was then extracted with ethyl acetate 100 ml. The organic solution was dried over sodium sulfate and concentrated to give 5.6 g of crude ester. To the ester, lithium hydroxide 2.5 g (60 mmol) and methanol (15 ml), THF (45 ml) and water (15 ml) was added. The mixture was stirred at room temperature overnight, concentrated to remove solvent, neutralized with 2N HCl to pH˜3, and extracted with ethyl acetate 40 ml. The organic layer was dried over sodium sulfate and concentrated to give 4.2 g of desired product.

### D. Synthesis of 2-(benzhydrylthio)acetic acid

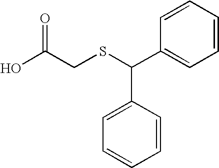

10 g of thiourea was dissolved in 57 ml of 48% HBr and 10 ml of water. The reaction mixture was heated to 60° C., and 20.2 g of benzhydrol was added. The temperature was increased to 90° C., cooled to room temperature. The crystals were filtered off and washed with water. The above crystals were added to 35 ml 30% sodium hydroxide. The mixture was heated to 70° C., and chloroacetic acid 11.44 g in 22 ml of water was added slowly. The mixture was refluxed for half an hour after the addition. The reaction mixture was then cooled to room temperature to give 25 g of desired product.

### E. Synthesis of 2-(benzhydrylsulfinyl)acetic acid

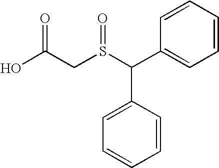

10 g of thiourea was dissolved in 57 ml of 48% HBr and 10 ml of water. The reaction mixture was heated to 60° C., and 20.2 g of benzhydrol was added. The temperature was increased to 90° C. and then cooled to room temperature. The crystals were filtered off and washed with water. The crystals were added to 35 ml 30% sodium hydroxide and the mixture was heated to 70° C., before 11.44 g chloroacetic acid in 22 ml of water was added slowly. The mixture was refluxed for half an hour after the addition. 14.3 ml hydrogen peroxide (30%) was added to the above solution within 3 hours at room temperature. 22 ml of water was then added and the reaction mixture was filtered. The filtrate was acidified with concentrated HCl (d=1.18). The resulting solid was filtered off and dried to give 13 g of the desired product.

### Example 2

### Synthesis of 2-(benzhydrylamino)-1-(4-benzhydrylpiperazin-1-yl)ethanone (compound no. 30)

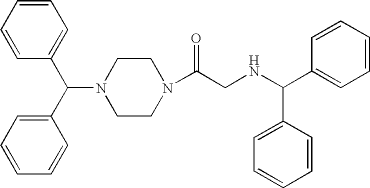

To a solution of 1-diphenylmethylpiperazine 0.125 g (0.5 mmol) dissolved in methylene chloride (5 ml) was added 2-(benzhydrylamino)acetic acid, 0.12 g (0.5 mmol) (synthesized according to Example 1(b)), EDC 0.191 g (1 mmole) and trace of 4-(dimethylamino)pyridine (DMAP), and the reaction mixture was stirred at room temperature overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.10 g of desired product.

### Example 3

### Synthesis of 2-(benzhydryl(methyl)amino)-1-(4-benzhydrylpiperazin-1-yl)ethanone (compound no. 2)

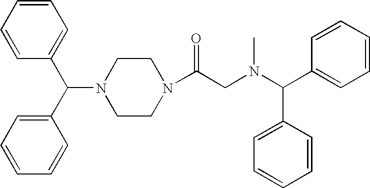

To a solution of 1-diphenylmethylpiperazine 0.125 g (0.5 mmol) dissolved in methylene chloride (5 ml) was added 2-(benzhydryl(methyl)amino)acetic acid, 0.13 g (0.5 mmol) (synthesized according to Example 1(a)), EDC 0.191 g (1 mmole) and trace of DMAP, and the reaction mixture was stirred at room temperature overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.11 g of desired product.

### Example 4

### Synthesis of 2-benzhydryl(methyl)amino-1-(4-(1-methyl-4-phenylpiperidine-4-carbonyl)piperazin-1-yl)ethanone (compound no. 8)

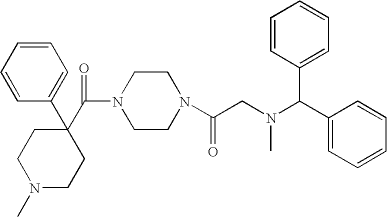

### A. Synthesis of 1-methyl-4-phenylpiperidine-4-carbonitrile

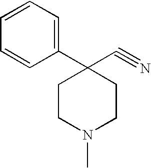

To a solution of mechlorethamine 4 g (25.6 mmol) and benzyl cyanide 4 g (34.2 mmol) in toluene (25 ml) was added sodium amide 2 g (51.2 mmol) at 40-50° C. in portions for 1 hour. The reaction mixture was heated to reflux for 2 hrs after the addition. The reaction mixture was then cooled to room temperature and washed with saturated sodium bicarbonate solution and brine, dried over sodium sulfate and concentrated. The residue was applied to flash column chromatography using methylene chloride and methanol (100:5) as eluents to give 3 g of desired product.

### B. Synthesis of 1-methyl-4-phenylpiperidine-4-carboxylic acid hydrochloride

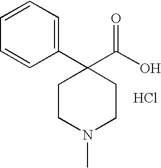

1-methyl-4-phenylpiperidine-4-carbonitrile 3 g (15 mmol) was refluxed with 6N HCl (40 ml) overnight. The reaction mixture was concentrated to remove water. The product (3.4 g) was obtained by heating and drying under vacuum in the oven and used in the next step without purification.

### C. Synthesis of (4-benzylpiperazin-1-yl)(1-methyl-4-phenylpiperidin-4-yl)methanone

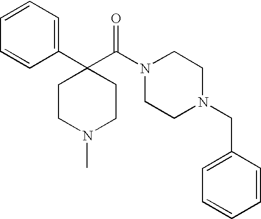

A solution of 1-methyl-4-phenylpiperidine-4-carboxylic acid hydrochloride, 1.35 g (5 mmol), benzyl piperazine 0.88 g (5 mmol), triethylamine 1 ml and EDC 1.91 g (10 mmol) and DMAP (trace) in 40 ml dichloromethane was stirred at room temperature overnight, and concentrated. Water was then added and the reaction product was extracted with ethyl acetate 2×50 ml. The combined organic solution was dried over sodium sulfate and concentrated. The residue was applied to flash column chromatography using methylene chloride and methanol (100:10) as eluents to give 1.5 g of desired product.

### D. Synthesis of (1-methyl-4-phenylpiperidin-4-yl)piperazin-1-yl)methanone

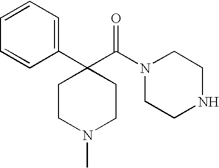

A mixture of (4-benzylpiperazin-1-yl)(1-methyl-4-phenylpiperidin-4-yl)methanone 1.5 g (4 mmol) and 20% PdOH/C in methanol (50 ml) was shaken under H₂ 50-60 psi for 18 hours. The mixture was then filtered and the solvent removed in vacuo to afford 0.9 g of desired product.

### E. Synthesis of 2-benzhydryl(methyl)amino-1-(4-(1-methyl-4-phenylpiperidine-4-carbonyl)piperazin-1-yl)ethanone

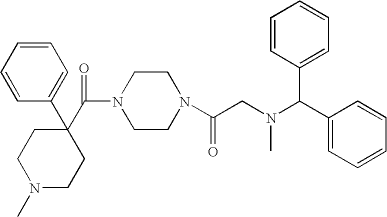

To a solution of (1-methyl-4-phenylpiperidin-4-yl)piperazin-1-yl)methanone, 0.15 g (0.5 mmol) dissolved in methylene chloride (5 ml) was added 2-(benzhydryl(methyl)amino)acetic acid, 0.130 g (0.5 mmol) (synthesized according to Example 1(a)), EDC 0.191 g (1 mmole) and trace of DMAP, and the reaction mixture was stirred at room temperature for overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.12 g of desired product.

### Example 5

### Synthesis of 2-(benzhydrylamino)-1-(4-((1-methylpiperidin-4-yl)methyl)piperazin-1-yl)ethanone (compound no. 9)

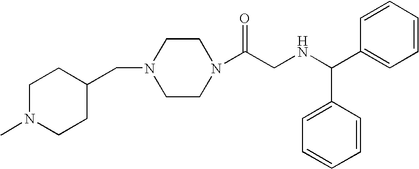

A solution of 1-((1-methylpiperidin-4-yl)methyl)piperazine 0.118 g (0.6 mmol), 2-(benzhydrylamino) acetic acid 0.159 g (0.66 mmol) (synthesized according to Example 1(b)), 1-ethyl-3-(3 dimethylaminopropyl) carbodiimide hydrochloride (EDC) 0.171 g (0.9 mmol) and catalytic amount of DMAP in methylene chloride 5 ml was stirred at room temperature overnight. The reaction mixture was concentrated, water was added and the product was extracted with ethyl acetate 2×50 ml. The combined organic solution was dried over sodium sulfate and concentrated. The residue was applied to flash column chromatography using ethyl acetate, methanol and triethylamine (85:10:5) as eluent to afford 0.21 g of desired product in 83.3% yield.

### Example 6

### Synthesis of 2-benzhydrylamino-1-(4-((1-methylpiperidin-4-yl)piperazine-1-yl)ethanone (compound no. 10)

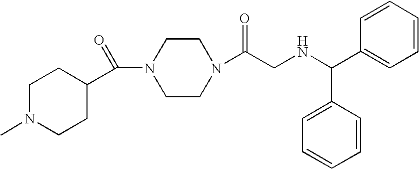

### A. Synthesis of Tert-butyl-4-((1-methylpiperidin-4-yl)(piperazin-1-yl)methanone carboxylate

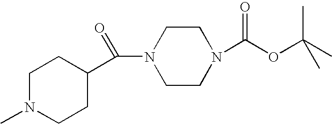

A solution of 1-methylpiperidine-4-carboxylic acid hydrochloride 0.7 g (4 mmol), Tert-butyl piperazine-1-carboxylate 0.613 g (3.3 mmol), triethylamine 1.12 ml (8 mmol), 1-ethyl-3-(3 dimethylaminopropyl) carbodiimide hydrochloride (EDC) 1.52 g (8 mmol) and catalytic amount of 4-(dimethylamino) pyridine in methylene chloride 20 ml were stirred at room temperature overnight. The reaction mixture was concentrated, and then water was added water before the reaction product was extracted with ethyl acetate 2×50 ml. The combined organic solution was dried over sodium sulfate and concentrated. The residue was applied to flash column chromatography using methylene chloride and methanol (100:10) as eluents to give 0.89 g of desired product in 85% yield.

### B. Synthesis of (1-methylpiperidin-4-yl)(piperazin-1-yl)methanone

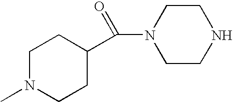

To a solution of Tert-butyl-4-((1-methylpiperidin-4-yl)(piperazin-1-yl)methanone carboxylate 0.8 g (2.6 mmol) in methylene chloride (20 ml) was added trifluoroacetic acid 5 ml and resulting mixture stirred at room temperature for 2 hours. The reaction mixture was concentrated, dissolved in methylene chloride and washed with saturated sodium bicarbonate and brine. The methylene chloride solution was dried over sodium sulfate and concentrated to give 0.5 g (quantitative) of desired product.

### C. Synthesis of 2-benzhydrylamino-1-(4-((1-methylpiperidin-4-yl)piperazine-1-yl)ethanone

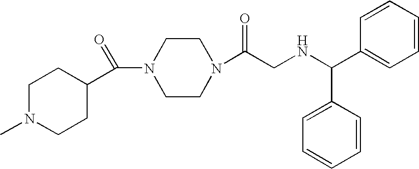

A solution of (1-methylpiperidin-4-yl)(piperazin-1-yl)methanone 0.105 g (0.5 mmol), 2-(benzhydrylamino) acetic acid 0.144 g (0.6 mmol) (synthesized according to Example 1(b)),1-ethyl-3-(3 dimethylaminopropyl) carbodiimide hydrochloride (EDC) 0.229 g (1.2 mmol) and a catalytic amount of 4-(dimethylamino) pyridine in methylene chloride (5 ml) were stirred at room temperature overnight. The reaction mixture was concentrated, and water was then added before the reaction product was extracted with ethyl acetate 2×20 ml. The combined organic solution was dried over sodium sulfate and concentrated. The residue was applied to flash column chromatography using methylene chloride and methanol (100:10) as eluents to give 0.156 g of desired product in 70% yield.

### Example 7

### Synthesis of (R)-2-(benzhydrylamino)-4-methyl-1-(4-((1-methylpiperidin-4-yl)methyl)piperazin-1-yl)pentan-1-one (compound 13)

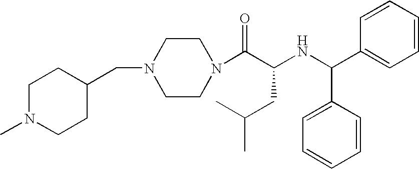

### A. Synthesis of tert-butyl (R)-4-methyl-1-(4-((1-methylpiperidin-4-yl)piperazin-1-yl-1-oxopentan-2-ylcarbamate

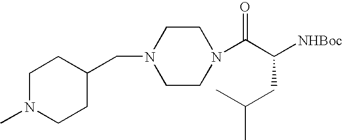

A solution of 1-((1-methylpiperidin-4-yl)methyl)piperazine 0.197 g (1 mmol) (prepared by LAH reduction of the amide from Example 6.B, using the method described in Example 11.D), Boc-L-Leucine 0.254 g (1.1 mmol), 1-ethyl-3-(3 dimethylaminopropyl) carbodiimide hydrochloride (EDC) 0.420 g (2.2 mmol) and catalytic amount of 4-(dimethylamino) pyridine in methylene chloride 5 ml were stirred at room temperature overnight. The reaction mixture was concentrated, water was added and the reaction product extracted with ethyl acetate 2×20 ml. The combined organic solution was dried over sodium sulfate and concentrated. The residue was applied to flash column chromatography using methylene chloride and methanol (95:5) as eluents to give 0.32 g of desired product in 78% yield.

### B. Synthesis of (R)-2-amino-4-methyl-1-(4-((1-methylpiperidin-4-yl)piperazin-1-yl)pentan-1-one trihydrochloride

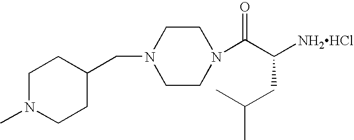

To a solution of tert-butyl (R)-4-methyl-1-(4-((1-methylpiperidin-4-yl)piperazin-1-yl)-1-oxopentan-2-ylcarbamate 0.32 g (0.8 mmol) in methylene chloride (20 ml) was added trifluoroacetic acid 5 ml and the resulting mixture was stirred at room temperature for 2 hours. The reaction mixture was then concentrated, re-dissolved in a saturated solution of HCl in diethyl ether and evaporated. The gummy residue thus obtained was again dissolved in ether/HCl solution and evaporated to yield about 0.3 g of the desired hydrochloride salt.

### C. Synthesis of (R)-2-(benzhydrylamino)-4-methyl-1-(4-((1-methylpiperidin-4-yl)methyl)piperazin-1-yl)pentan-1-one

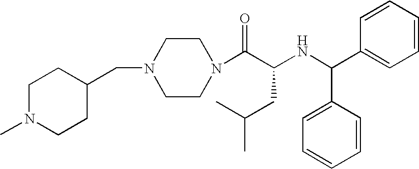

A solution of (R)-2-amino-4-methyl-1-(4-((1-methylpiperidin-4-yl)piperazin-1-yl)pentan-1-one hydrochloride 0.3 g (1 mmol), benzophenone imine 0.2 ml (1 mmol) and triethylamine 0.2 ml in dichloroethane 25 ml was refluxed for 15 h. Solid ammonium chloride was filtered off and DCE was evaporated. The residue was further re-dissolved in methylene chloride and washed with brine (2×20 ml). The organic solution was dried over sodium sulfate and evaporated under vacuum to yield 0.48 g (quantitative) of benzhydryl Schiff's base. The crude product was subsequently dissolved in methanol 20 ml and sodium borohydride 0.5 g (15 mmol) was then added. The reaction was stirred at RT for 24 h, concentrated and residue further re-dissolved in methylene chloride, washed with saturated sodium bicarbonate, and brine. The organics were dried over sodium sulfate and evaporated under vacuo. The residue was applied to flash column chromatography using ethyl acetate, methanol and triethylamine (85:10:5) as eluents to give 0.3 g of desired product in 71% yield.

### Example 8

### Synthesis of 2-(benzhydryl(methylamino)-1-(4-((1-methylpiperidin-4-yl)(phenyl)methyl)piperazin-1-yl)ethanone (compound no. 14)

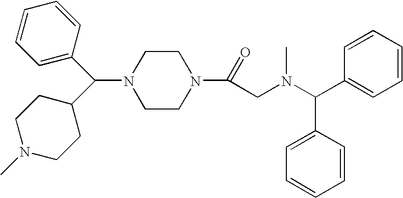

### A. Synthesis of (1-methylpiperidin-4-yl)(phenyl)methanone

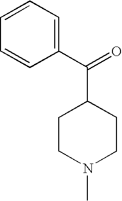

1-Methylpiperidine-4-carboxylic acid hydrochloride salt 10 g (55.7 mmol) was added to thionyl chloride (25 ml) and stirred at room temperature until the solid dissolved completely. The reaction mixture was stirred for another 20 minutes and concentrated. The product was used for the next step without further purification.

To a cooled suspension of anhydrous aluminum chloride (20 g, 75 mmol) in benzene 30 ml at 0° C. was added 1-methylpiperidine-4-carboxylic acid chloride in small portions and the resulting mixture was refluxed for 3 hours. The reaction mixture was then cooled down by adding to ice water. The organic phase was discarded. The aqueous solution was washed with 2×50 ml ethyl ether, basified with potassium hydroxide pellet slowly to pH >10 and extracted with ethyl ether 4×50 ml. The combined ethereal solution was dried over sodium sulfate and concentrated to give 9.5 g of desired product in 84% yield.

### B. Synthesis of (1-methylpiperidin-4-yl)(phenyl)methanol

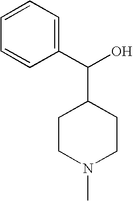

To a solution of 1-methylpiperidin-4-yl phenyl methanone 1.02 g (5 mmol) in 30 ml methanol was added in small portions sodium borohydride 0.378 g (10 mmol). The reaction mixture was stirred at room temperature for two hours and then concentrated. Water was added and the reaction product was then extracted with methylene chloride 2×50 ml. The combined organic solution was dried over sodium sulfate and concentrated to give 1 g of desired product in 98% yield.

### C. Synthesis of 4-(chloro)phenylmethyl-1-methylpiperidine

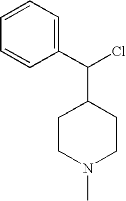

To a solution of 4-chlorophenyl 1-methylpiperidin-4-yl methanol 1.2 g (5.85 mmol) in toluene (5 ml) was added thionyl chloride (0.5 ml) dropwise. The resulting mixture was stirred at room temperature overnight. The mixture was then made alkaline with NaOH solution and extracted with ethyl acetate (3×40). The combined organic solution was dried and concentrated to give 1.2 g of desired product.

### D. Synthesis of 1-(phenyl)(1-methylpiperidin-4-yl)methyl)piperazine

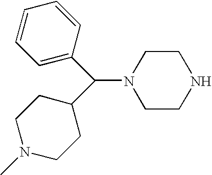

A mixture of 4-(chloro(phenyl)methyl)-1-methylpiperidine (1.2 g, 5.38 mmol) in butanone (10 ml), anhydrous piperazine (1.9 g, 21.52 mmol), anhydrous K₂CO₃ (0.74 g, 5.38 mmol) and KI (0.89 g, 5.38 mmol) was refluxed under nitrogen for 18 hours. The mixture was then cooled and filtered and the solvent removed in vacuo. The residue was dissolved in CH₂Cl₂ (50 ml) and washed with water (30 ml). Drying and removal of the solvent followed by chromatography (CH₂Cl₂:CH₃OH:NH₄OH 90:10:0.5) afforded the desired product 1-(phenyl)(1-methylpiperidin-4-yl)methyl)piperazine in 70% yield.

### E. Synthesis of 2-(benzhydryl(methylamino)-1-(4-((1-methylpiperidin-4-yl)(phenyl)methyl)piperazin-1-yl)ethanone

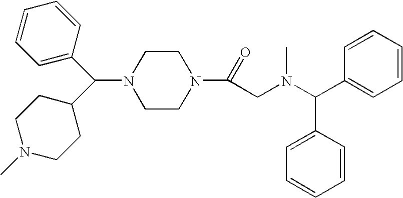

To a solution of compound 1-(phenyl)(1-methylpiperidin-4-yl)methyl)piperazine 0.15 g (0.5 mmol) dissolved in methylene chloride (5 ml) was added 2-(benzhydryl(methyl)amino)acetic acid 0.130 g (0.5 mmol) (synthesized according to Example 1(a)), EDC 0.191 g (1 mmol) and trace of DMAP, and the reaction mixture was stirred at room temperature for overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.12 g of desired product.

### Example 9

### Synthesis of 1-(4-benzhydrylpiperazin-1-yl)-2-(phenylamino)ethanone (compound no. 16)

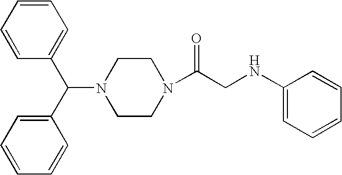

### A. Synthesis of 1-(4-benzhydrylpiperazin-1-yl)-2-chloroethanone

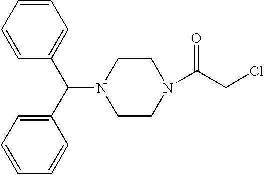

Diphenylmethyl piperazine (4 g, 15.8 mmol) and triethylamine (3.31 ml, 23.8 mmol) were combined in dry THF (50 ml). Chloroacetyl chloride (1.39 ml, 17.4 mmol) was added to the reaction via syringe. The reaction was stirred at room temperature for 16 h under a N₂ atmosphere. The reaction was concentrated in vacuo and the crude residue dissolved in CH₂Cl₂ and washed with saturated NaHCO₃. The organic layer was dried (Na₂SO₄) and concentrated under reduced pressure. The desired product was isolated as an oil after purification by silica gel chromatography (5% EtOAc/CH₂Cl₂, R_{f}=0.5). MS (C19H₂₁N₂OCl+1) 329.3.

### B. Synthesis of 1-(4-benzhydrylpiperazin-1-yl)-2-(phenylamino)ethanone

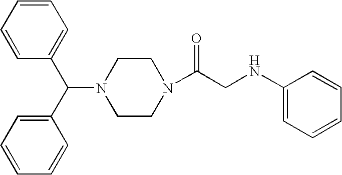

1-Diphenylmethyl-4-(chloroacetyl)piperazine (200 mg, 0.60 mmol), aniline (62.3 mg, 0.66 mmol), and potassium carbonate (126 mg, 0.91 mmol) were combined in acetonitrile (20 ml) and refluxed for 16 h. The reaction was concentrated in vacuo and the crude purified by silica gel chromatography (5% EtOAc/CH₂Cl₂, RF 0.3) to yield the product as an oil. The free amine was converted to the HCl salt by dissolving the product in CH₂Cl₂ followed by the addition of HCl in diethyl ether (100 mg, 30%). MS (C25H₂₇N_{3O}1) 386.3.

### Example 10

### Synthesis of 2-(benzhydryloxy)-1-(4-benzyhydrylpiperazin-1-yl)ethanone (compound no. 30)

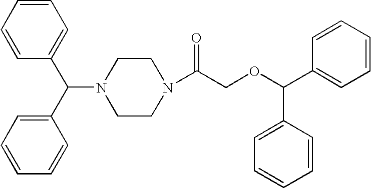

To a solution of 1-diphenylmethylpiperazine 0.125 g (0.5 mmol) dissolved in methylene chloride (5 ml) was added 2-(benzhydryloxy)acetic acid 0.12 g (0.5 mmol) (synthesized according to Example 1(c)), EDC 0.191 g (1 mmole) and trace of DMAP, and the reaction mixture was stirred at room temperature overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.11 g of desired product.

### Example 11

### Synthesis of 2-(benzhydrylsulfinyl)-1-(2,6-dimethyl-4-((1-methylpiperidin-4-yl)methyl)piperazin-1-yl)ethanone (compound no. 31)

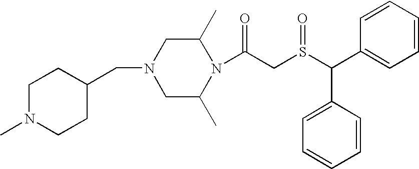

### A. Synthesis of 1-(tert-butoxycarbonyl)piperidin-4-carboxylic methyl ester

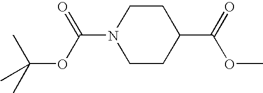

A solution of methyl isonipecotate 7.2 g (50 mmol), Boc anhydride 12 g (55 mmol), triethylamine 7 ml and in 80 ml methanol was stirred at room temperature overnight, concentrated, water was added and the reaction product was extracted with ethyl acetate 2×50 ml. The combined organic solution was dried over sodium sulfate and concentrated. The residue was applied to flash column chromatography using ethyl acetate:petroleum ether (3:1) as eluents to give 12 g of desired product.

### B. Synthesis of 1-(tert-butoxycarbonyl)piperidin-4-carboxylic acid

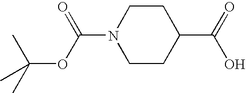

A mixture of 1-(tert-butoxycarbonyl)piperidin-4-carboxylic acid methyl ester 2.42 g (10 mmol) and LiOH.3H2O 1.26 g (30 mmol) in THF (45 ml) water (15 ml) and methanol (15 ml) was stirred at room temperature overnight. The mixture was then concentrated to remove the solvent. The residue was adjust to pH˜2 with 2N HCl and extracted with ethyl acetate (2×40 ml). The combined organic solution was dried with sodium sulfate and concentrated to give 2.3 g of desired acid.

### C. Synthesis of tert-butyl-4-(3,5-dimethylpiperazine-1-carbonyl)piperidine-1-carboxylate

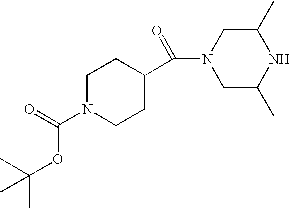

A solution of 1-(tert-butoxycarbonyl)piperidin-4-carboxylic acid 2.3 g (10 mmol), 2,6-dimethylpiperazine 1.14 g (10 mmol), and EDC 3.82 g (20 mmol) and DMAP (trace) in 20 ml dichloromethane was stirred at room temperature overnight. The mixture was concentrated, and water was added before the reaction product was extracted with ethyl acetate 2×50 ml. The combined organic solution was dried over sodium sulfate and concentrated. The residue was applied to flash column chromatography using methylene chloride and methanol (100:5) as eluents to give 1.9 g of the desired product.

### D. Synthesis of 3,5-dimethyl-1-((1-methylpiperidin-4-yl)methyl)piperazine

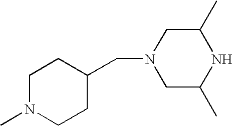

To a solution of tert-butyl-4-(3,5-dimethylpiperazine-1-carbonyl)piperidine-1-carboxylate, 1.9 g (5.8 mmol) in THF (50 ml) was added LiAlH₄ 0.440 g (11.6 mmol) in portions. The resulting mixture was stirred at room temperature overnight. The mixture was quenched with ethyl acetate and methanol then made alkaline with 10% NaOH solution and extracted with ethyl acetate (3×40). The combined organic solution was dried and concentrated to give 1.2 g of desired product.

### E. Synthesis of 2-(benzhydrylsulfinyl)-1-(2,6-dimethyl-4-((1-methylpiperidin-4-yl)methyl)piperazin-1-yl)ethanone

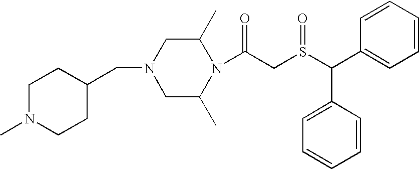

A solution of 3,5-dimethyl-1-((1-methylpiperidin-4-yl)methyl)piperazine 0.125 g (0.55 mmol), 2-(benzhydrylsulfinyl)acetic acid 0.164 g (0.6 mmol) synthesized according to Example 1(e), 1-ethyl-3-(3 dimethylaminopropyl) carbodiimide hydrochloride (EDC) 0.229 g (1.2 mmol) and catalytic amount of 4-(dimethylamino) pyridine in methylene chloride 5 ml were stirred at room temperature overnight. The reaction mixture was concentrated and water was added before the reaction mixture was extracted with ethyl acetate 2×20 ml. The combined organic solution was dried over sodium sulfate and concentrated. The residue was applied to flash column chromatography using methylene chloride and methanol (100:10) as eluents to give 0.192 g of desired product in 71% yield.

### Example 12

### Synthesis of 2-(benzhydryloxy)-1-(2,6-dimethyl-4-((tetrahydro-2H-pyran-4-yl)methyl)piperazin-1-yl)ethanone (compound 32)

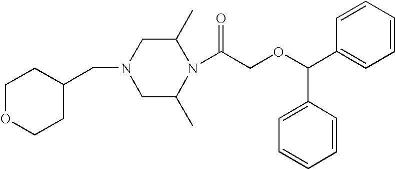

### A. Synthesis of tetrahydro-2H-pyran-4-carboxylic acid

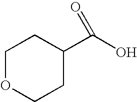

A mixture of methyl tetrahydro-2H-pyran-4-carboxylate, 7.2 g (50 mmol) and LiOH.3H₂O 6.3 g (150 mmol) in THF (50 ml) water (15 ml) and methanol (15 ml) was stirred at room temperature overnight. The mixture was then concentrated to remove the solvent. The residue was adjusted to pH˜2 with 2N HCl and extracted with ethyl acetate (2×40 ml). The combined organic solution was dried with sodium sulfate and concentrated to give 6.0 g of desired acid.

### B. Synthesis of (3,5-dimethylpiperazin-1-yl)(tetrahydro-2H-pyran-4-yl)methanone

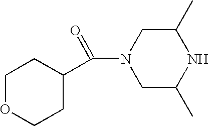

A solution of 2,6-dimethylpiperazine 1.14 g (10 mmol, tetrahydro-2H-pyran-4-carboxylic acid 1.3 g (10 mmol), 1-ethyl-3-(3 dimethylaminopropyl) carbodiimide hydrochloride (EDC) 3.82 g (20 mmol) and a catalytic amount of 4-(dimethylamino) pyridine in methylene chloride 20 ml were stirred at room temperature overnight. The reaction mixture was concentrated and water was added before the reaction product was extracted with ethyl acetate 2×20 ml. The combined organic solution was dried over sodium sulfate and concentrated. The residue was applied to flash column chromatography using methylene chloride and methanol (100:10) as eluents to give 1.76 g of desired product in 78% yield.

### C. Synthesis of 3,5-dimethyl-1-(tetrahydro-2H-pyran-4-yl)methyl)piperazine

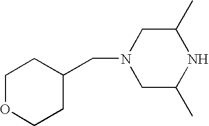

To a solution of (3,5-dimethylpiperazin-1-yl)(tetrahydro-2H-pyran-4-yl)methanone 1.5 g (7 mmol) in THF (50 ml) was added LiAlH₄ 0.5 g (13 mmol) in portions. The resulting mixture was stirred at room temperature over night. The mixture was quenched with ethyl acetate and methanol then made alkaline with 10% NaOH solution and extracted with ethyl acetate (3×40). The combined organic solution was dried and concentrated to give 1.2 g of desired product.

### D. Synthesis of 2-(benzhydryloxy)-1-(2,6-dimethyl-4-((tetrahydro-2H-p ran-4-yl)methyl)piperazin-1-yl)ethanone

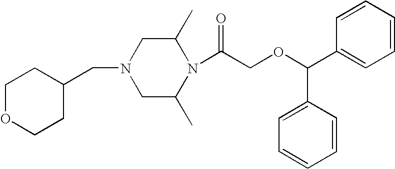

A solution of 3,5-dimethyl-1-(tetrahydro-2H-pyran-4-yl)methyl)piperazine, 0.124 g (0.55 mmol), 2-(benzhydryloxy)acetic acid 0.145 g (0.6 mmol) (synthesized according to Example 1(c)), 1-ethyl-3-(3 dimethylaminopropyl) carbodiimide hydrochloride (EDC) 0.230 g (1.2 mmol) and catalytic amount of 4-(dimethylamino) pyridine in methylene chloride 5 ml were stirred at room temperature overnight. The reaction mixture was concentrated and water was added before the reaction product was extracted with ethyl acetate 2×20 ml. The combined organic solution was dried over sodium sulfate and concentrated. The residue was applied to flash column chromatography using methylene chloride and methanol (100:10) as eluents to give 0.135 g of desired product in 54% yield.

### Example 13

### Synthesis of 2-(benzhydrylamino)-1-(4-((tetrahydro-2H-pyran-4-yl)methyl)piperazin-1-yl)ethanone (compound no. 38)

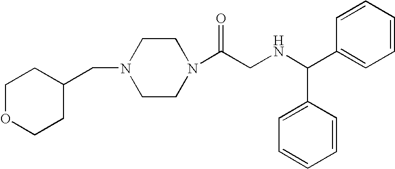

### A. Synthesis of (4-benzylpiperazin-1-yl)(tetrahydro-2H-pyran-4-yl)methanone

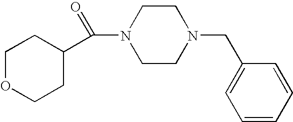

A solution of 4-benzyl piperazine 1.76 g (10 mmol), tetrahydro-2H-pyran-4-carboxylic acid 1.3 g (10 mmol), 1-ethyl-3-(3 dimethylaminopropyl) carbodiimide hydrochloride (EDC) 3.82 g (20 mmol) and catalytic amount of 4-(dimethylamino) pyridine in methylene chloride (20 ml) were stirred at room temperature overnight. The reaction mixture was concentrated, water was added and the reaction product was extracted with ethyl acetate 2×20 ml. The combined organic solution was dried over sodium sulfate and concentrated. The residue was applied to flash column chromatography using ethyl acetate and petroleum ether (1:1) as eluents to give 2.24 g of desired product in 78% yield.

### B. Synthesis of 1-benzyl-4-(tetrahydro-2H-pyran-4-yl)piperazine

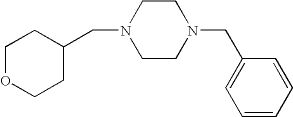

To a solution of (4-benzylpiperazin-1-yl)(tetrahydro-2H-pyran-4-yl)methanone 2.5 g (8.7 mmol) in THF (40 ml) was added LiAlH₄ 0.5 g (13 mmol) in portions. The resulting mixture was stirred at room temperature over night. The mixture was quenched with ethyl acetate and methanol then made alkaline with 10% NaOH solution and extracted with ethyl acetate (3×40). The combined organic solution was dried and concentrated to give 2.2 g of desired product.

### C. Synthesis of (tetrahydro-2H-pyran-4-yl)piperazine

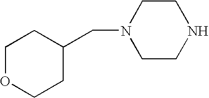

To a solution of 1-benzyl-4-((tetrahydro-2H-pyran-4-yl)piperazine, 2.0 g (7.5 mmol) in MeOH 40 ml and formic acid 83% 0.5 ml was added 10% Pd/C 0.4 g and exposed to 50 psi hydrogen overnight. The reaction mixture was filtered. The filtrate was concentrated to give 1.28 g of desired product in 93% yield.

### D. Synthesis of 2-(benzhydrylamino)-1-(4-((tetrahydro-2H-pyran-4-yl)methyl)piperazin-1-yl)ethanone

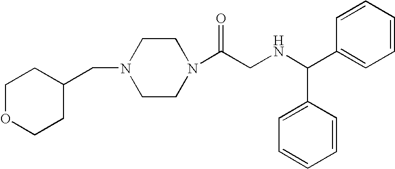

A solution of (tetrahydro-2H-pyran-4-yl)piperazine, 0.110 g (0.6 mmol), 2-(benzhydrylamino)acetic acid 0.160 g (0.66 mmol) (synthesized according to Example 1(b)), 1-ethyl-3-(3 dimethylaminopropyl) carbodiimide hydrochloride (EDC) 0.252 g (1.32 mmol) and catalytic amount of 4-(dimethylamino) pyridine in methylene chloride (5 ml) were stirred at room temperature overnight. The reaction mixture was concentrated, water was added and the reaction product extracted with ethyl acetate 2×20 ml. The combined organic solution was dried over sodium sulfate and concentrated. The residue was applied to flash column chromatography using methylene chloride and methanol (100:5) as eluents to give 0.12 g of desired product in 50% yield.

### Example 14

### Synthesis of 1-(4-benzhydrylpiperazin-1-yl)-2-(4-benzhydrylthio)ethanone (compound no. 44)

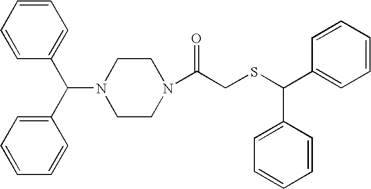

To a solution of 1-diphenylmethylpiperazine 0.125 g (0.5 mmol) dissolved in methylene chloride (5 ml) was added 2-(benzhydrylthio)acetic acid 0.13 g (0.5 mmol) (synthesized according to Example 1(d)), EDC 0.191 g (1 mmole) and trace of DMAP, and the reaction mixture was stirred at room temperature for overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.10 g of desired product.

### Example 15

### Synthesis of 1-(4-benzhydrylpiperazin-1-yl)-2-(4-benzhydrylsulfinyl)ethanone (compound no. 45)

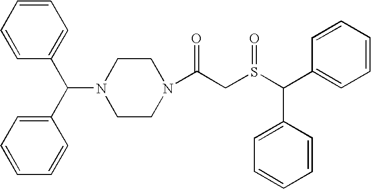

To a solution of 1-diphenylmethylpiperazine 0.125 g (0.5 mmol) dissolved in methylene chloride (5 ml) was added 2-(benzhydrylsulfinyl)acetic acid 0.138 g (0.5 mmol) (synthesized according to Example 1(e)), EDC 0.191 g (1 mmole) and trace of DMAP, and the reaction mixture was stirred at room temperature overnight. The reaction mixture was concentrated and dissolved in ethyl acetate (10 ml) and washed with saturated sodium bicarbonate solution and brine, dried over sodium sulfate and concentrated. The residue was applied to flash column chromatography using methylene chloride and methanol (100:10) as eluents to give 0.13 g of desired product.

### Example 16

### Synthesis of 2-benzhydrylamino-1-(4-benzhydryl-3-hydroxymethylpiperazin-1-yl)ethanone (compound no. 49)

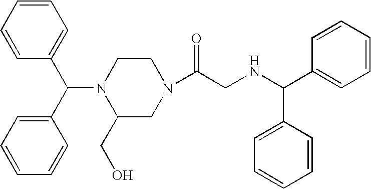

### A. Synthesis of ethyl-1,4-dibenzylpiperazine-2-carboxylate dihydrochloride

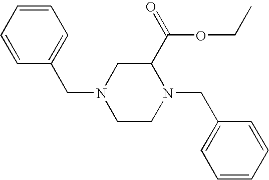

N,N′-dibenzyl ethyl diamine (26.28 g, 109 mmol) and triethylamine (22.08 g, 218 mmol) were combined in toluene (500 mL). Ethyl 2,3-dibromopropionate (28.42 g, 111 mmol) was added to the reaction mixture which was then refluxed for 5 h. The solvent was removed in vacuo leaving an oil which was dissolved in methanol and subsequently added to a solution of HCl in methanol. The reaction was concentrated and the resulting solid was dried under hi-vac to yield the desired product (37 g, 82%).

### B. Synthesis of ethyl piperazine-2-carboxylate dihydrochloride

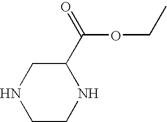

Ethyl 1,4-dibenzylpiperazine-2-carboxylate dihydrochloride (10 g, 24 mmol) was dissolved in methanol (200 mL) to which was added Pd/C (2 g, 10% w/w). The reaction was placed on a Parr hydrogenator under 50 psi hydrogen gas for 16 h. Upon completion of the reaction, the mixture was filtered and the filtrate concentrated to yield the desired product as a solid (5.53 g, quant.)

### C. Synthesis of 1-tert-butyl 3-ethyl piperazine-1,3-dicarboxylate

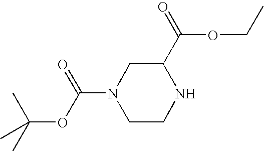

Ethyl piperazine-2-carboxylate dihydrochloride (6.8 g, 29 mmol) and triethylamine (8.91 g, 89 mmol) were combined in dichloromethane (150 mL) and cooled in an ice-brine bath to 0° C. Boc anhydride (6.42 g, 29 mmol) in dichloromethane (50 mL) was added to the reaction over 1 h. After the Boc₂O solution was added, the reaction was quenched on ice. The organic layer was removed and the aqueous layer extracted with dichloromethane (3×50 mL). The pooled organic fractions were dried (Na₂SO₄) and concentrated in vacuo. The residue was purified by silica gel chromatography (1:1 dichloromethane:ethyl acetate to ethyl acetate (R_{f} 0.5)) to yield the desired product as a clear oil (6.25 g, 83%).

### D. Synthesis of 3-ethyl-1-tert-butyl-4-diphenylmethylpiperazine-1,3-dicarboxylate

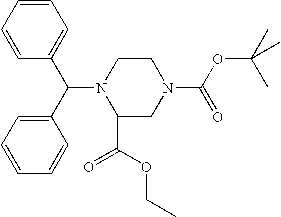

1-tert-butyl 3-ethyl piperazine-1,3-dicarboxylate (6.25 g, 24 mmol), bromo diphenylmethane (8.97 g, 36 mmol), and potassium carbonate (10.03 g, 72 mmol) were combined in acetonitrile (300 mL). The reaction was then refluxed for 3 h. The crude reaction was then filtered before washing the resulting solid with additional portions of acetonitrile. The filtrate was concentrated under reduced pressure and the resulting residue was purified by silica gel chromatography (3:1 pet. ether:dichloromethane to 1:9 ethyl acetate:dichloromethane (R_{f} 0.7)) to yield the desired product as a white solid (8.39 g, 82%).

### E. Synthesis of 1-tert-butyl-4-(diphenylmethyl)-3-(hydroxymethyl)piperazine-1-carboxylate

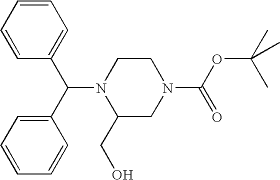

3-Ethyl 1-tert-butyl 4-diphenylmethylpiperazine-1,3-dicarboxylate (4.78 g, 11.2 mmol) was dissolved in diethyl ether (225 mL) under a N₂ atmosphere. Lithium borohydride (0.736 g, 33.8 mmol) was added to the reaction with stirring. Methanol (1.08 g, 33.8 mmol) was slowly added to the reaction via syringe. The reaction was refluxed for 1 h after which it was cooled and quenched in 10% NaOH (75 mL). The diethyl ether layer was separated and the aqueous layer was extracted with ethyl acetate (3×75 mL). The pooled organic fractions were dried (Na₂SO₄) and concentrated. The product was used without further purification (3.21 g, 75%).

### F. Synthesis of 1-tert-butyl-4-(diphenylmethyl)-3-(tert-butyl dimethyl silyloxymethyl)piperazine-1-carboxylate

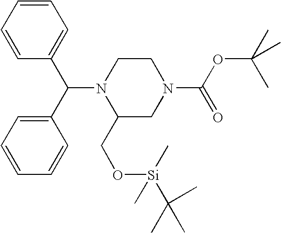

1-tert-Butyl 4-(diphenylmethyl)-3-(hydroxymethyl)piperazine-1-carboxylate (2 g, 5.2 mmol) and imidazole (1.42 g, 20.8 mmol) were dissolved in dichloromethane (60 mL). tert-Butyl dimethyl silyl chloride (1.58 g, 10.4 mmol) was added to the reaction portionwise. The reaction was refluxed for 2 h under N₂. The reaction was quenched with 1 N NaOH (20 mL). The aqueous layer was washed with dichloromethane (3×75 mL). The organic fractions were then combined, dried (Na₂SO₄) and concentrated in vacuo. The desired product was isolated after silica gel chromatography (1:1 pet. ether:dichloromethane (R_{f} 0.5) to dichloromethane (R_{f} 0.9)) as a clear oil (2.41 g, 93%).

### G. Synthesis of 1-diphenylmethyl-2-(tert-butyl dimethyl silyloxymethyl)piperazine

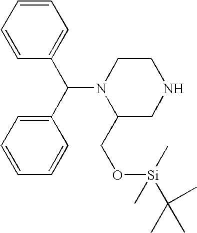

1-tert-butyl 4-(diphenylmethyl)-3-(tert-butyl dimethyl silyloxymethyl)piperazine-1-carboxylate (2.41 g, 4.8 mmol) was dissolved in dry dichloromethane (80 mL). Dry zinc bromide, (10.92 g, 48.5 mmol) was added. The reaction was stirred under N₂ for 24 h. The reaction was quenched by adding water (30 mL) and stirring for 1 h after which no solid remained. The aqueous layer was extracted with dichloromethane (3×80 mL). The pooled organic fractions were dried (Na₂SO₄) and concentrated. The product was purified via silica gel chromatography (1:1:18 methanol:triethylamine:ethyl acetate (R_{f} 0.5)) as a thick, clear oil (1.68 g, 68%).

### H. Synthesis of 2-benzhydrylamino-1-[4-benzhydryl-3-(tert-butyl dimethyl silyl hydroxymethyl)piperazin-1-yl]ethanone

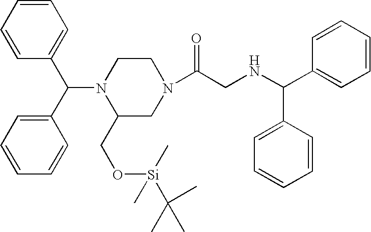

1-Diphenylmethyl-2-(tert-butyl dimethyl silyl hydroxymethyl)piperazine (616 mg, 1.55 mmol), 2-(benzhydrylamino)acetic acid (0.450 g, 1.86 mmol) (synthesized according to Example 1(b)), EDC (595 mg, 3.10 mmol), and DMAP (cat.) were added to methylene chloride (16 mL). The reaction was stirred for 72 h at room temperature. The reaction was then diluted with dichloromethane (60 mL) and washed with 1 N NaOH (40 mL). The aqueous layer was washed with dichloromethane (3×60 mL). The combined organic fractions were dried (Na₂SO₄) and concentrated under reduced pressure. The final product was purified by silica gel chromatography (1:9 ethyl acetate:dichloromethane (R_{f} 0.5)) as viscous oil (200 mg, 21%).

### I. Synthesis of 2-benzhydrylamino-1-(4-benzhydryl-3-hydroxymethylpiperazin-1-yl)ethanone

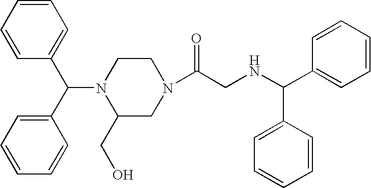

2-benzhydrylamino-1-[4-benzhydryl-3-(tert-butyl dimethyl silyloxymethyl)piperazin-1-yl]ethanone (200 mg, 0.32 mmol) was dissolved in THF (20 mL). Tetrabutyl ammonium fluoride (1M in THF, 645 mL, 0.64 mmol) was added via syringe. The reaction was stirred at room temperature for 3 h. Upon completion, the reaction was concentrated under reduced pressure. The crude was applied to a silica gel column to yield the free base of the product as a thick oil. The product was dissolved in dichloromethane and precipitated with HCl/diethyl ether as the HCl salt (153.5 mg, 82%).

### Example 17

### Synthesis of 2-benzhydrylamino-1-(4-benzhydryl-3-methoxymethylpiperazin-1-yl)ethanone (compound no. 53)

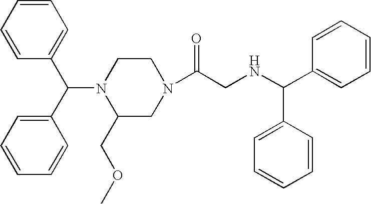

### A. Synthesis of 1-tert-butyl-4-(diphenylmethyl)-3-(methoxymethyl)piperazine-1-carboxylate

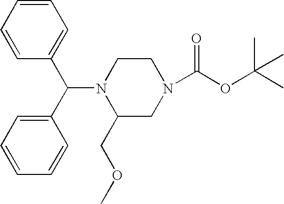

1-tert-Butyl 4-(diphenylmethyl)-3-(hydroxymethyl)piperazine-1-carboxylate (2 g, 5.2 mmol) and silver (II) oxide (2.42 g, 10.4 mmol) were combined in acetonitrile (40 mL). Methyl iodide (4.38 g, 30.9 mmol) was added via syringe. The reaction was refluxed for 16 h. Upon completion, the reaction was filtered and the filtrate concentrated in vacuo. The product was purified by silica gel chromatography (1:19 ethyl acetate:dichloromethane (R_{f} 0.8)) and isolated as a gummy solid (1.44 g, 70%).

### B. Synthesis of 1-diphenylmethyl-2-(methoxymethyl)piperazine

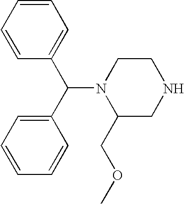

1-tert-butyl 4-(diphenylmethyl)-3-(methoxymethyl)piperazine-1-carboxylate (1.41 g, 3.6 mmol) and zinc bromide (8.01 g, 36 mmol) were stirred in dry dichloromethane (50 mL) for 24 h at room temperature. Water (20 mL) was added to the reaction which was stirred for an additional 1 h. The aqueous layer was separated and washed with dichloromethane (3×50 mL). The pooled organic fractions were dried (Na₂SO₄) and concentrated in vacuo. The product was purified via silica gel chromatography (1:1:18 methanol:triethylamine:ethyl acetate (R_{f} 0.5)) as a thick, yellow oil (0.91 g, 85%).

### C. Synthesis of 2-benzhydrylamino-1-(4-benzhydryl-3-methoxymethylpiperazin-1-yl)ethanone

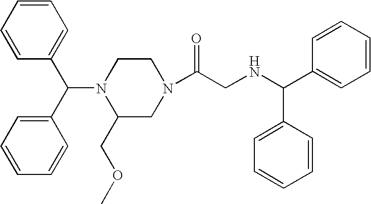

1-Diphenylmethyl-2-(methoxymethyl)piperazine (210 mg, 0.71 mmol), 2-(benzhydrylamino)acetic acid (205 mg, 0.85 mmol) (synthesized according to Example 1(b)), EDC (272 mg, 1.42 mmol), and DMAP (cat.) were added to dry dichloromethane (5 mL). The reaction was stirred for 48 h at room temperature. The reaction was diluted with dichloromethane (60 mL) and washed with 1 N NaOH (40 mL). The aqueous layer was washed with dichloromethane (3×60 mL). The combined organic fractions were dried (Na₂SO₄) and concentrated under reduced pressure. The final product was purified by silica gel chromatography (1:4 ethyl acetate:dichloromethane (R_{f} 0.8)) as viscous oil. The product was dissolved in dichloromethane and precipitated with HCl/diethyl ether as the HCl salt (185 mg, 44%).

### Example 18

Following the procedures set forth above, the following compounds listed in Table 1 below were prepared. Mass spectrometry was employed with the final compound and at various stages throughout the synthesis as a confirmation of the identity of the product obtained. For the mass spectrometric analysis, samples were prepared at an approximate concentration of 1 μg/mL in acetonitrile with 0.1% formic acid. Samples were then manually infused into an Applied Biosystems API3000 triple quadrupole mass spectrometer and scanned in Q1 in the range of 50 to 700 m/z.

TABLE 1

| | | | Mass |
|---|---|---|---|
| Cmpd | | | Spec |
| No. | Name | Structure | (m/z) |
| 1 | 2-benzhydrylamino-1-(4- benzhydrylpiperazin-1-yl)ethanone | 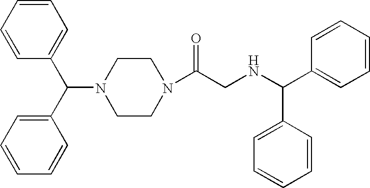 | 476.3 |
| 2 | 2-benzhydryl(methyl)amino-1-(4- benzhydrylpiperazin-1-yl)ethanone | 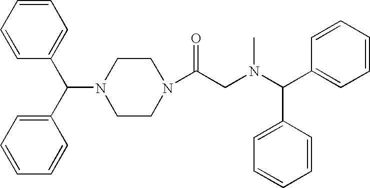 | 490.4 |
| 3 | 2-benzhydryl(methyl)amino-1-(4-((4- chlorophenyl)(phenyl)methyl)piperazin- 1-yl)ethanone | 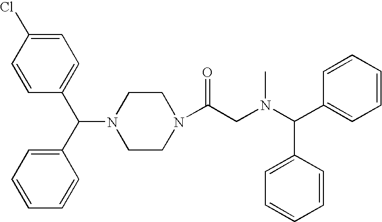 | 524.5 |
| 4 | 2-benzhydryl(methyl)amino-1-(4-((3- chlorophenyl)(phenyl)methyl)piperazin- 1-yl)ethanone | 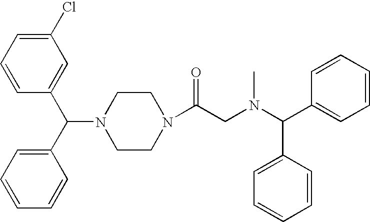 | 524.5 |
| 5 | 2-benzhydryl(methyl)amino-1-(4-((2- chlorophenyl)(phenyl)methyl)piperazin- 1-yl)ethanone | 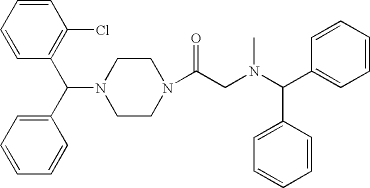 | 524.5 |
| 6 | 2-benzhydryl(methyl)amino-1-(4-((4- fluorophenyl)(phenyl)methyl)piperazin- 1-yl)ethanone | 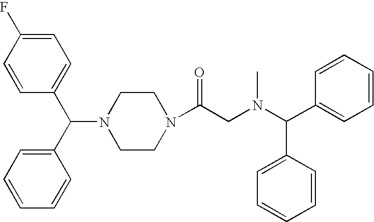 | 508.5 |
| 7 | 2-benzhydryl(methyl)amino-1-(4- (phenyl(3- trifluoromethyl)phenyl)methyl)piperazin- 1-yl)ethanone | 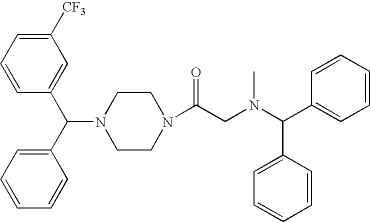 | 558.4 |
| 8 | 2-benzhydryl(methyl)amino-1-(4-(1- methyl-4-phenylpiperidine-4- carbonyl)piperazin-1-yl)ethanone | 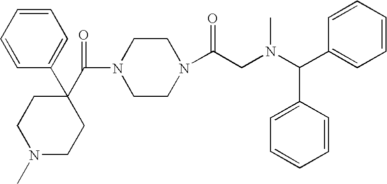 | 525.5 |
| 9 | 2-benzhydrylamino-1-(4-((1- methylpiperidin-4-yl)piperazine-1- yl)ethanone | 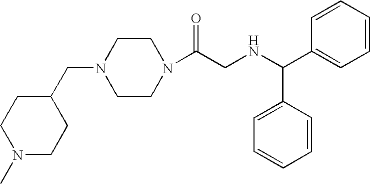 | 421.4 |
| 10 | 2-benzhydrylamino-1-(4-(1- methylpiperidine-4-carbonyl)piperazin- 1-yl)ethanone | 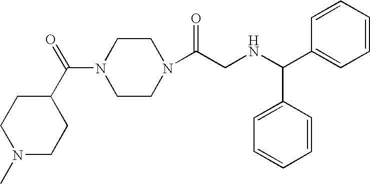 | 435.5 |
| 11 | 2-benzhydryl(methyl)amino-1-(4-(1- methylpiperidine-4-carbonyl)piperazin- 1-yl)ethanone | 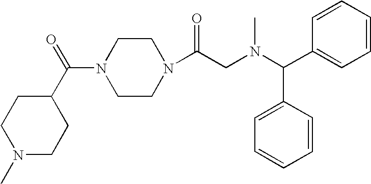 | 449.5 |
| 12 | 2-benzhydryl(methyl)amino-1-(4-((1- methylpiperidin-4-yl)piperazin-1-yl) ethanone | 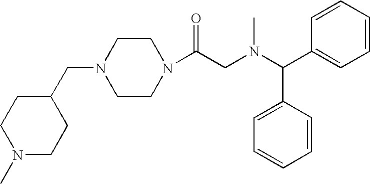 | 435.7 |
| 13 | (R)-2-(benzhydrylamino)-4-methyl-1-(4- ((1-methylpiperidin-4-yl)methyl) piperazin-1-yl)pentan-1-one | 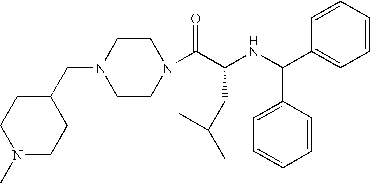 | 477.5 |
| 14 | 2-benzhydryl(methyl)amino-1-(4-((1- methylpiperidin-4-yl)(phenyl) methyl)piperazin-1-yl)ethanone | 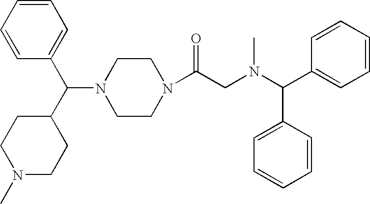 | 511.4 |
| 15 | 2-benzhydryl(methyl)amino-1-(4-((4- fluorophenyl)(1-methylpiperidin-4- yl)methyl)piperazin-1-yl)ethanone | 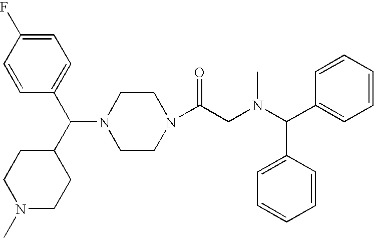 | 529.5 |
| 16 | 1-(4-benzhydrylpiperazin-1-yl)-2- phenylamino)ethanone | 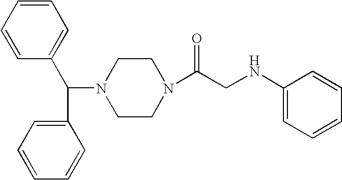 | 386.3 |
| 17 | 1-(4-benzhydrylpiperazin-1-yl)-2-(4- chlorophenylamino)ethanone | 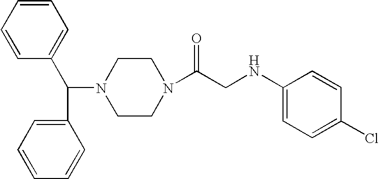 | 420.2 |
| 18 | 1-(4-benzhydrylpiperazin-1-yl)-2-(3- chlorophenylamino)ethanone | 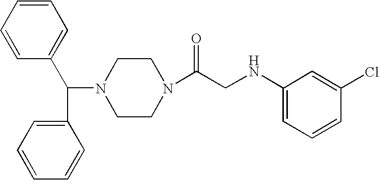 | 420.4 |
| 19 | 1-(4-benzhydrylpiperazin-1-yl)-2-(2- chlorophenylamino)ethanone | 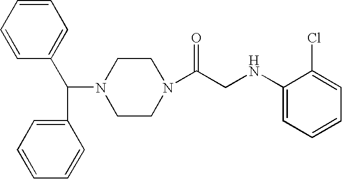 | 420.4 |
| 20 | 1-(4-benzhydrylpiperazin-1-yl)-2-(2,4- difluorophenylamino)ethanone | 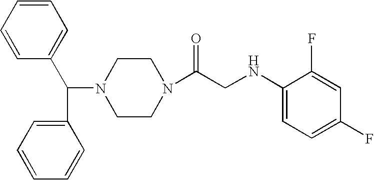 | 422.4 |
| 21 | 1-(4-benzhydrylpiperazin-1-yl)-2-(3,5- difluorophenylamino)ethanone | 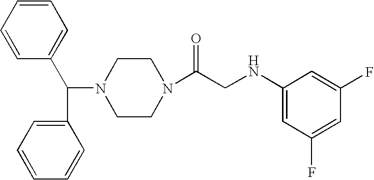 | 422.4 |
| 22 | 1-(4-benzhydrylpiperazin-1-yl)-2-(3,5- dichlorophenylamino)ethanone | 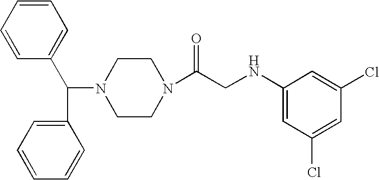 | 454.4 |
| 23 | 1-(4-benzhydrylpiperazin-1-yl)-2-(3,5- dimethylphenylamino) ethanone | 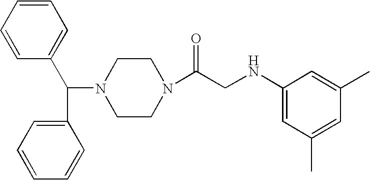 | 414.5 |
| 24 | 1-(4-benzhydrylpiperazin-1-yl)-2-(4- fluorophenylamino)ethanone | 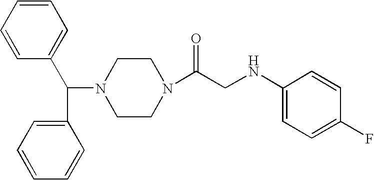 | 404.3 |
| 25 | 1-(4-benzhydrylpiperazin-1-yl)-2-(3,4,5- trimethoxyphenylamino) ethanone | 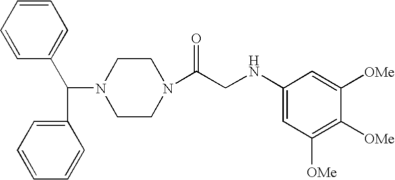 | 476.4 |
| 26 | 1-(4-benzhydrylpiperazin-1-yl)-2-(2,4- dichlorophenoxy)ethanone | 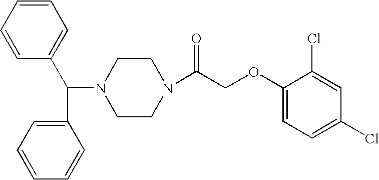 | 455.5 |
| 27 | 1-(4-benzhydrylpiperazin-1-yl)-2- (methyl(phenyl)amino)ethanone | 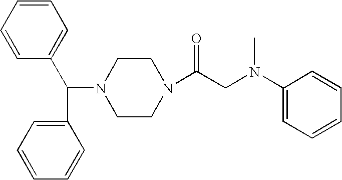 | 400.4 |
| 28 | 2-benzhydryl(methyl)amino-1-(4-(1-(1- methylpiperidin-4-yl)-1- phenylethyl)piperazin-1-yl)ethanone | 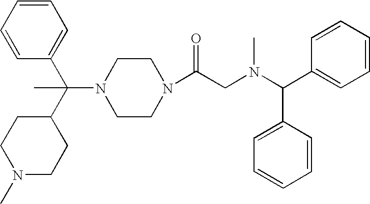 | 525.5 |
| 29 | 2-(benzhydrylamino)-1-(4-(bis(4- fluorophenyl)methyl)piperazin-1- yl)ethanone | 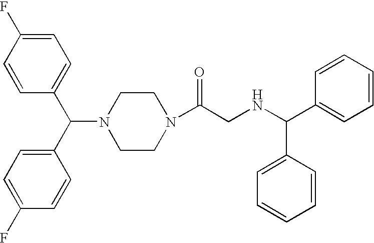 | 512.3 |
| 30 | 2-(benzhydryloxy)-1-(4-benzhydryl piperazin-1-yl)ethanone | 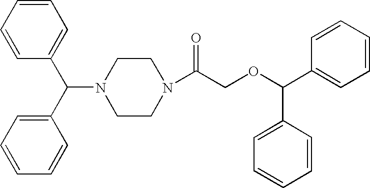 | 477.4 |
| 31 | 2-(benzhydrylsulfinyl)-1-(2,6-dimethyl- 4-((1-methylpiperidin-4-yl)methyl) piperazin-1-yl)ethanone | 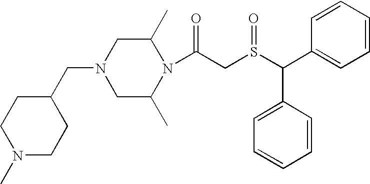 | 482.0 |
| 32 | 2-(benzhydryloxy)-1-(2,6-dimethyl-4- ((tetrahydro-2H-pyran-4-yl)methyl) piperazin-1-yl)ethanone | 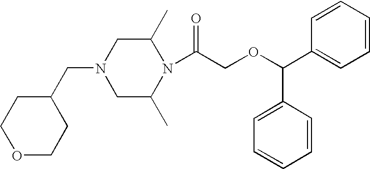 | 437.5 |
| 33 | 2-(benzhydrylthio)-1-(2,6-dimethyl-4- ((tetrahydro-2H-pyran-4-yl)methyl) piperazin-1-yl)ethanone | 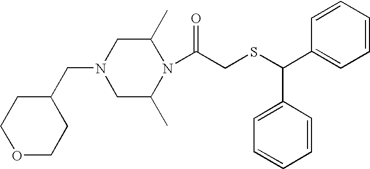 | 453.3 |
| 34 | 2-(benzhydrylsulfinyl)-1-(2,6-dimethyl- 4-((tetrahydro-2H-pyran-4-yl)methyl) piperazin-1-yl)ethanone | 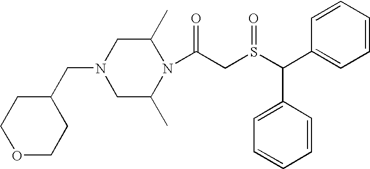 | 469.4 |
| 35 | 2-(benzhydryloxy)-1-(4-((tetrahydro- 2H-pyran-4-yl)methyl)piperazin-1- yl)ethanone | 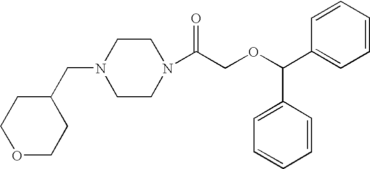 | 409.4 |
| 36 | 2-(benzhydrylthio)-1-(4-((tetrahydro- 2H-pyran-4-yl)methyl)piperazin-1- yl)ethanone | 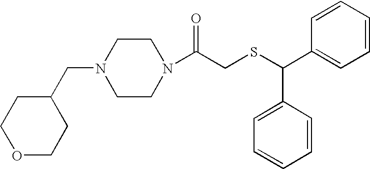 | 425.3 |
| 37 | 2-(benzhydrylsulfinyl)-1-(4- ((tetrahydro-2H-pyran-4-yl)methyl) piperazin-1-yl)ethanone | 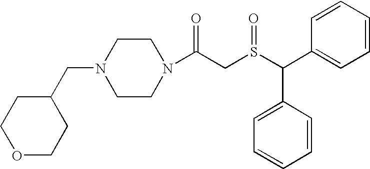 | 441.4 |
| 38 | 2-(benzhydrylamino)-1-(4-((tetrahydro- 2H-pyran-4-yl)methyl)piperazin-1- yl)ethanone | 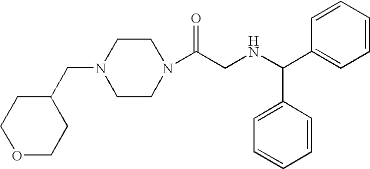 | 408.4 |
| 39 | 2-(benzhydryloxy)-1-(4-((2- chloropyridin-4-yl)methyl)piperazin-1- yl)ethanone | 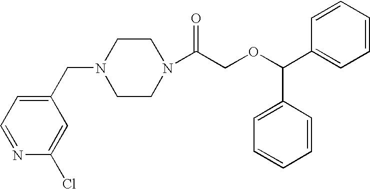 | 436.3 |
| 40 | 2-(benzhydryloxy)-1-(4-((2-chloro-6- methylpyridin-4-yl)methyl)piperazin-1- yl)ethanone | 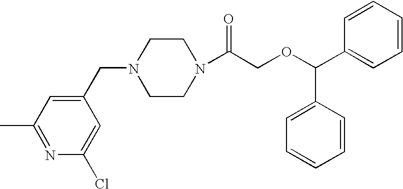 | 450.3 |
| 41 | 2-(benzhydryloxy)-1-(4-((1- methylpiperidin-4-yl)methyl)piperazin- 1-yl)ethanone | 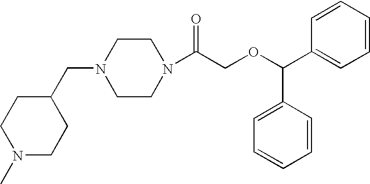 | 422.3 |
| 42 | 2-(benzhydrylthio)-1-(4-((1- methylpiperidin-4-yl)methyl) piperazin-1-yl)ethanone | 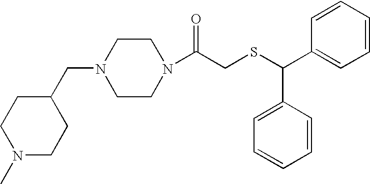 | 438.4 |
| 43 | 2-(benzhydrylsulfinyl)-1-(4-((1- methylpiperidin-4-yl)methyl) piperazin-1-yl)ethanone | 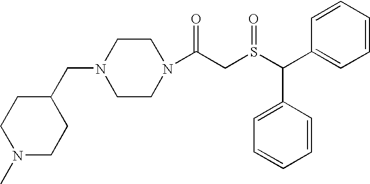 | 454.4 |
| 44 | 1-(4-benzhydrylpiperazin-1-yl)-2- (benzhydrylthio)ethanone | 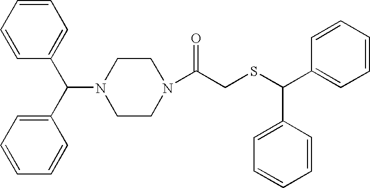 | 493.4 |
| 45 | 1-(4-benzhydrylpiperazin-1-yl)-2- (benzhydrylsulfinyl)ethanone | 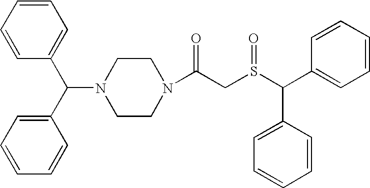 | 509.3 |
| 46 | 2-(benzhydryloxy)-1-(4-(bis(4- fluorophenyl)methyl)piperazin-1- yl)ethanone | 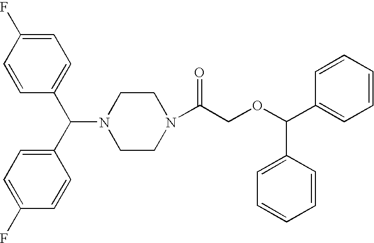 | 513.3 |
| 47 | 2-(benzhydrylthio)-1-(4-(bis(4- fluorophenyl)methyl)piperazin-1- yl)ethanone | 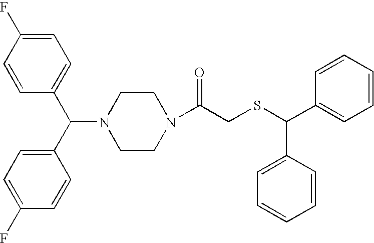 | 529.4 |
| 48 | 2-(benzhydrylsulfinyl)-1-(4-(bis(4- fluorophenyl)methyl)piperazin-1- yl)ethanone | 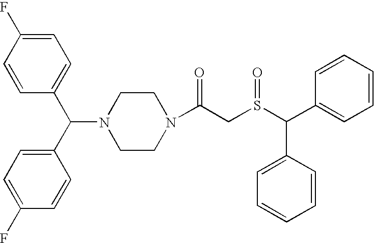 | 545.4 |
| 49 | 2-benzhydrylamino-1-(4-benzhydryl-3- hydroxymethylpiperazin-1-yl)ethanone | 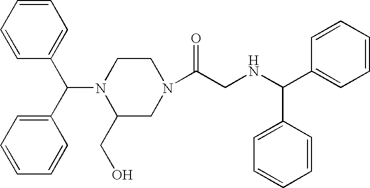 | 506.4 |
| 50 | 2-(benzhydryloxy)-1-(4-benzhydryl-3- hydroxymethyl piperazin-1-yl)ethanone | 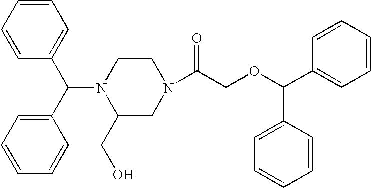 | 507.1 |
| 51 | 1-(4-benzhydryl-3- hydroxymethylpiperazin-1-yl)-2- (benzhydrylsulfinyl)ethanone | 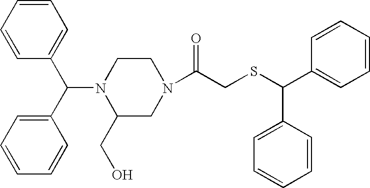 | 523.3 |
| 52 | 1-(4-benzhydryl-3-hydroxymethyl piperazin-1-yl)-2-(benzhydryl sulfinyl)ethanone | 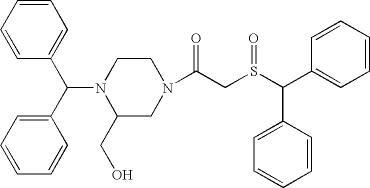 | 539.3 |
| 53 | 2-benzhydrylamino-1-(4-benzhydryl-3- methoxymethyl piperazin-1-yl)ethanone | 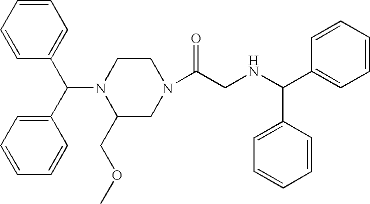 | 520.5 |
| 54 | 2-(benzhydryloxy)-1-(4-benzhydryl-3- methoxymethyl piperazin-1-yl)ethanone | 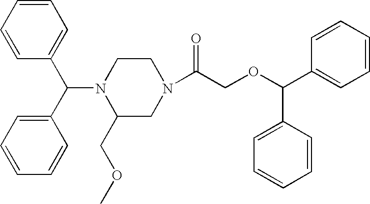 | 521.5 |
| 55 | 1-(4-benzhydryl-3- methoxymethylpiperazin-1-yl)-2- (benzhydrylsulfinyl)ethanone | 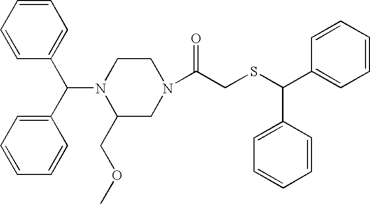 | 537.3 |
| 56 | 1-(4-benzhydryl-3-methoxymethyl piperazin-1-yl)-2-(benzhydryl sulfinyl)ethanone | 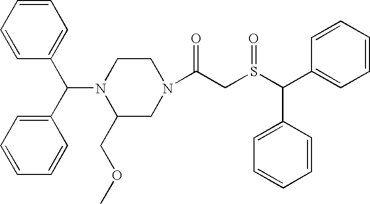 | 553.4 |

In a similar manner, but substituting 4-aminopiperidine for piperazine, the compounds above where Z is CHNH, rather than N can be prepared.

### Example 19

### N-Type Channel Blocking Activities of Various Invention Compounds

A. Transformation of HEK Cells:
N-type calcium channel blocking activity was assayed in human embryonic kidney cells, HEK 293, stably transfected with the rat brain N-type calcium channel subunits (α_{1B}+α₂δ+β_{1b} cDNA subunits). Alternatively, N-type calcium channels (α_{1B}+α₂δ+β_{1b} cDNA subunits), L-type channels (α_{1C}+α₂δ+β_{1b} cDNA subunits) and P/Q-type channels (α_{1A}+α₂δ+β_{1b} cDNA subunits) were transiently expressed in HEK 293 cells. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/1 mM EDTA and plated at 10% confluency on glass coverslips. At 12 hours the medium was replaced and the cells transiently transfected using a standard calcium phosphate protocol and the appropriate calcium channel cDNA's. Fresh DMEM was supplied and the cells transferred to 28° C./5% CO₂. Cells were incubated for 1 to 2 days prior to whole cell recording.

B. Measurement of Inhibition

Whole cell patch clamp experiments were performed using an Axopatch 200B amplifier (Axon Instruments, Burlingame, Calif.) linked to a personal computer equipped with pCLAMP software. The external and internal recording solutions contained, respectively, 5 mM BaCl₂, 10 mM MgCl₂, 10 mM HEPES, 40 mM TEACl, 10 mM glucose, 87.5 mM CsCl (pH 7.2) and 108 mM CsMS, 4 mM MgCl₂, 9 mM EGTA, 9 mM HEPES (pH 7.2). Currents were typically elicited from a holding potential of −80 mV to +10 mV using Clampex software (Axon Instruments). Typically, currents were first elicited with low frequency stimulation (0.067 Hz) and allowed to stabilize prior to application of the compounds. The compounds were then applied during the low frequency pulse trains for two to three minutes to assess tonic block, and subsequently the pulse frequency was increased to 0.2 Hz to assess frequency dependent block. Data were analyzed using Clampfit (Axon Instruments) and SigmaPlot 4.0 (Jandel Scientific).

Specific data obtained for N-type channels are shown in Table 2 below.

TABLE 2
N-type Calcium Channel Block

| Compound | IC₅₀ @ 0.067 Hz (μM) | IC₅₀ @ 0.2 Hz (μM) |
|---|---|---|
| 1 | 0.14 | 0.08 |
| 2 | 0.52 | 0.31 |
| 3 | 2.45 | 0.85 |
| 4 | 1.24 | 0.84 |
| 5 | >4.67 | >2.94 |
| 6 | 0.17 | 0.12 |
| 7 | 1.46 | 0.67 |
| 9 | 4.00 | 2.50 |
| 10 | >4.97 | >2.64 |
| 11 | >11.84 | 7.92 |
| 12 | 7.40 | 2.07 |
| 14 | 0.55 | 0.44 |
| 16 | 4.30 | 0.80 |
| 17 | >7.60 | >4.60 |
| 18 | 1.29 | 1.03 |
| 21 | 1.40 | 0.70 |
| 22 | 2.40 | 0.98 |
| 25 | 2.30 | 1.50 |
| 26 | 0.25 | 0.19 |
| 29 | 0.27 | 0.14 |
| 30 | 0.49 | 0.27 |
| 32 | 1.00 | 0.64 |
| 34 | >6.60 | >4.70 |
| 35 | 8.30 | 1.30 |
| 38 | 9.53 | 4.91 |
| 40 | 1.20 | 0.74 |
| 41 | >4.80 | >3.20 |
| 43 | 4.26 | 1.34 |
| 44 | 0.60 | 0.27 |
| 45 | 0.29 | 0.20 |
| 46 | 0.92 | 0.43 |
| 47 | 4.92 | 3.23 |
| 48 | 0.22 | 0.16 |
| 49 | 0.49 | 0.29 |
| 50 | 0.40 | 0.23 |
| 52 | 1.23 | 0.97 |
| 53 | 0.25 | 0.15 |
| 54 | 0.33 | 0.21 |
| 56 | 0.18 | 0.13 |

### Example 20

### T-Type Channel Blocking Activities of Various Invention Compounds

Standard patch-clamp techniques were employed to identify blockers of T-type currents. Briefly, previously described HEK cell lines stably expressing human α_{1G} T-type channels were used for all the recordings (passage #: 4-20, 37° C., 5% CO₂). To obtain T-type currents, plastic dishes containing semi-confluent cells were positioned on the stage of a ZEISS AXIOVERT S100 microscope after replacing the culture medium with external solution (see below). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (see below for internal solution).

TABLE 3
External Solution 500 ml - pH 7.4, 265.5 mOsm

| Salt | Final mM | Stock M | Final ml | |
|---|---|---|---|---|
| CsCl | 132 | 1 | 66 | |
| CaCl₂ | 2 | 1 | 1 | |
| MgCl₂ | 1 | 1 | 0.5 | |
| HEPES | 10 | 0.5 | 10 | |
| glucose | 10 | — | 0.9 | grams |

TABLE 4
Internal Solution 50 ml - pH 7.3 with CsOH, 270 mOsm

| Salt | Final mM | Stock M | Final ml |
|---|---|---|---|
| Cs-Methanesulfonate | 108 | — | 1.231 gr/50 ml |
| MgCl2 | 2 | 1 | 0.1 |
| HEPES | 10 | 0.5 | 1 |
| EGTA-Cs | 11 | 0.25 | 2.2 |
| ATP | 2 | 0.2 | 0.025 |
| | | | (1 aliquot/2.5 ml) |

T-type currents were reliably obtained by using two voltage protocols:
(1) “non-inactivating”, and
(2) “inactivation”

In the non-inactivating protocol, the holding potential is set at −110 mV and with a pre-pulse at −100 mV for 1 second prior to the test pulse at 40 mV for 50 ms. In the inactivation protocol, the pre-pulse is at approximately −85 mV for 1 second, which inactivates about 15% of the T-type channels.

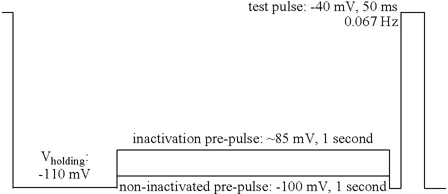

Test compounds were dissolved in external solution, 0.1-0.01% DMSO. After ˜10 min rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. IC₅₀ values are shown for various compounds of the invention in Table 5.

TABLE 5
T-type Calcium Channel Block

| Compound | IC₅₀ @ −80 mV (μM) |
|---|---|
| 1 | 0.08 |
| 2 | 2.90 |
| 6 | 2.00 |
| 26 | 1.26 |
| 29 | 9.69 |
| 30 | 0.59 |
| 46 | 0.35 |
| 48 | 0.31 |
| 49 | 0.37 |
| 53 | 0.49 |

The results from Table 5 can be used in isolation to indicate compounds that act as efficient T-type calcium channel blockers. Alternatively, the results from Table 5 can be used in conjunction with the results from Table 2 to indicate compounds that are effective in blocking both N-type and T-type calcium channels or are selective for N-type calcium channels.

### Example 21

### Activity of Invention Compounds in Formalin-Induced Pain Model

The effects of intrathecally delivered compounds of the invention on the rat formalin model can also be measured. The compounds can be reconstituted to stock solutions of approximately 10 mg/ml in propylene glycol. Typically eight Holtzman male rats of 275-375 g size are randomly selected per test article.

The following study groups are used, with test article, vehicle control (propylene glycol) and saline delivered intraperitoneally (IP):

TABLE 6
Formalin Model Dose Groups

| Test/Control Article | Dose | Route | Rats per group |
|---|---|---|---|
| Compound | 30 mg/kg | IP | 6 |
| Propylene glycol | N/A | IP | 4 |
| Saline | N/A | IP | 7 |

N/A = Not Applicable

Prior to initiation of drug delivery baseline behavioral and testing data can be taken. At selected times after infusion of the Test or Control Article these data can then be again collected.

On the morning of testing, a small metal band (0.5 g) is loosely placed around the right hind paw. The rat is placed in a cylindrical Plexiglas chamber for adaptation a minimum of 30 minutes. Test Article or Vehicle Control Article is administered 10 minutes prior to formalin injection (50 μl of 5% formalin) into the dorsal surface of the right hindpaw of the rat. The animal is then placed into the chamber of the automated formalin apparatus where movement of the formalin injected paw is monitored and the number of paw flinches tallied by minute over the next 60 minutes (Malmberg, A. B., et al., Anesthesiology (1993) 79:270-281).

Results can be presented as Maximum Possible Effect±SEM, where saline control=100%.

### Example 22

### Spinal Nerve Ligation Model of Neuropathic Pain

Spinal nerve ligation (SNL) injury was induced using the procedure of Kim and Chung, (Kim, S. H., et al., Pain (1992) 50:355-363) in male Sprague-Dawley rats (Harlan; Indianapolis, Ind.) weighing 200 to 300 grams. Anesthesia was induced with 2% halothane in O₂ at 2 L/min and maintained with 0.5% halothane in O₂. After surgical preparation of the rats and exposure of the dorsal vertebral column from L₄ to S₂, the L₅ and L₆ spinal nerves were tightly ligated distal to the dorsal root ganglion using 4-0 silk suture. The incision was closed, and the animals were allowed to recover for 5 days. Rats that exhibited motor deficiency (such as paw-dragging) or failure to exhibit subsequent tactile allodynia were excluded from further testing. Sham control rats underwent the same operation and handling as the experimental animals, but without SNL.

The assessment of tactile allodynia consisted of measuring the withdrawal threshold of the paw ipsilateral to the site of nerve injury in response to probing with a series of calibrated von Frey filaments. Each filament was applied perpendicularly to the plantar surface of the ligated paw of rats kept in suspended wire-mesh cages. Measurements were taken before and after administration of drug or vehicle. Withdrawal threshold was determined by sequentially increasing and decreasing the stimulus strength (“up and down” method), analyzed using a Dixon non-parametric test (Chaplan S. R., et al., J Pharmacol Exp Ther (1994) 269:1117-1123), and expressed as the mean withdrawal threshold.

The method of Hargreaves and colleagues (Hargreaves, K., et al., Pain (1988) 32:77-8) can be employed to assess paw-withdrawal latency to a thermal nociceptive stimulus. Rats are allowed to acclimate within a plexiglas enclosure on a clear glass plate maintained at 30° C. A radiant heat source (i.e., high intensity projector lamp) is then activated with a timer and focused onto the plantar surface of the affected paw of nerve-injured or carrageenan-injected rats. Paw-withdrawal latency can be determined by a photocell that halted both lamp and timer when the paw is withdrawn. The latency to withdrawal of the paw from the radiant heat source is determined prior to carrageenan or L5/L5 SNL, 3 hours after carrageenan or 7 days after L5/L6 SNL but before drug and after drug administration. A maximal cut-off of 40 seconds is employed to prevent tissue damage. Paw withdrawal latencies can be thus determined to the nearest 0.1 second. Reversal of thermal hyperalgesia is indicated by a return of the paw withdrawal latencies to the pre-treatment baseline latencies (i.e., 21 seconds). Anti nociception is indicated by a significant (p<0.05) increase in paw withdrawal latency above this baseline. Data is converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

Compound 49 was administered orally in propylene glycol solution at a dose of 300 mg/kg and complete reversal of allodynia was observed.

1. A method to treat a condition modulated by calcium ion channel activity, which method comprises administering to a subject in need of such treatment an amount of the compound of formula (1) effective to ameliorate said condition, wherein said compound is of the formula: 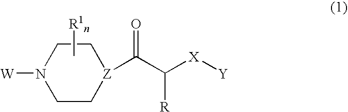
or a pharmaceutically acceptable salt or conjugates thereof
wherein X is NR², O, S, S═O, or SO₂;
Z is N or CHNR²;
Y is CR³Ar₂ or Ar;
W is CR³Ar₂, CR³AB or C═OA,
wherein at least one of W and Y is CR³Ar₂;
each Ar is independently an optionally substituted aromatic or heteroaromatic ring;
A is an optionally substituted aromatic or heteroaromatic ring, or an optionally substituted carbocyclic or heterocyclic ring;
B is halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or B may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
R is H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R may be an optionally substituted group selected from alkyl (1-8C), alkenyl (2-8C), or alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
each R¹ is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R¹ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
each R² is independently H, or an optionally substituted group selected from alkyl (1-8C), alkenyl (2-8C) and alkynyl (2-8C);
each R³ is independently H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R³ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), and C6-C12-aryl-C1-C8-alkyl;
n is 0-4; and
one or more optional substituents may be on one or more of Ar, A or B wherein, when the substituents on Ar, A or B is on an aromatic or heteroaromatic group, each optional substituent is independently selected from halo, CN, NO₂, CF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10); or each optional substituent may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl; and
when the substituents on A or B is on a non-aromatic group, each substituent is independently selected from the group consisting of ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10); or each substituent may be independently selected from alkyl (1-8C), alkenyl (2-8C), or alkynyl (2-8C), heteroalkyl (2-8C), heteroalkenyl (2-8C), heteroalkynyl (2-8C), aryl (6-10C), heteroaryl (5-12C), O-aryl (6-10C), O-heteroaryl (5-12C) and C6-C12-aryl-C1-C8-alkyl.

2. The method of claim 1 wherein said condition is modulated by N-type calcium channel activity.

3. The method of claim 1 wherein said condition is chronic or acute pain, mood disorders, neurodegenerative disorders, gastrointestinal disorders, genitourinary disorders, neuroprotection, metabolic disorders, cardiovascular disease, epilepsy, diabetes, prostate cancer, sleep disorders, Parkinson's disease, schizophrenia or male birth control.

4. The method of claim 3 wherein said condition is chronic or acute pain.

5. The method of claim 1, wherein Z is N.

6. The method of claim 1, wherein W is optionally substituted benzhydryl.

7. The method of claim 1, wherein Y is optionally substituted benzhydryl.

8. The method of claim 1, wherein W is CR³AB wherein A is 1-methylpiperidin-4-yl.

9. The method of claim 8 wherein R³ is H.

10. The method of claim 9 wherein B is H or optionally substituted phenyl.

11. The method of claim 1, wherein Y is unsubstituted benzhydryl.

12. The method of claim 1, wherein Y is optionally substituted phenyl.

13. The method of claim 1, wherein X is NH or NMe.

14. The method of claim 1, wherein each Ar is phenyl.

15. The method of claim 1, wherein R is H.

16. The method of claim 1, wherein n is 0 or 1 or 2.

17. The method of claim 1, wherein Ar, A and B are unsubstituted.

18. The method of claim 1, wherein the compound is selected from the group consisting of:
2-benzhydrylamino-1-(4-benzhydrylpiperazin-1-yl)ethanone;
2-benzhydryl(methyl)amino-1-(4-benzhydrylpiperazin-1-yl)ethanone;
2-benzhydryl(methyl)amino-1-(4-((4-chlorophenyl)(phenyl)methyl)piperazin-1-yl)ethanone;
2-benzhydryl(methyl)amino-1-(4-((3-chlorophenyl)(phenyl)methyl)piperazin-1-yl)ethanone;
2-benzhydryl(methyl)amino-1-(4-((2-chlorophenyl)(phenyl)methyl)piperazin-1-yl)ethanone;
2-benzhydryl(methyl)amino-1-(4-((4-fluorophenyl)(phenyl)methyl)piperazin-1-yl)ethanone;
2-benzhydryl(methyl)amino-1-(4-(phenyl(3-trifluoromethyl)phenyl)methyl)piperazin-1-yl)ethanone;
2-benzhydryl(methyl)amino-1-(4-(1-methyl-4-phenylpiperidine-4-carbonyl)piperazin-1-yl)ethanone;
2-benzhydrylamino-1-(4-((1-methylpiperidin-4-yl)piperazine-1-yl)ethanone;
2-benzhydrylamino-1-(4-(1-methylpiperidine-4-carbonyl)piperazin-1-yl)ethanone;
2-benzhydryl(methyl)amino-1-(4-(1-methylpiperidine-4-carbonyl)piperazin-1-yl)ethanone;
2-benzhydryl(methyl)amino-1-(4-((1-methylpiperidin-4-yl)piperazin-1-yl)ethanone;
(R)-2-(benzhydrylamino)-4-methyl-1-(4-((1-methylpiperidin-4-yl)methyl)piperazin-1-yl)pentan-1-one;
2-benzhydryl(methyl)amino-1-(4-((1-methylpiperidin-4-yl)(phenyl)methyl)piperazin-1-yl)ethanone;
2-benzhydryl(methyl)amino-1-(4-((4-fluorophenyl)(1-methylpiperidin-4-yl)methyl)piperazin-1-yl)ethanone;
1-(4-benzhydrylpiperazin-1-yl)-2-(phenylamino)ethanone;
1-(4-benzhydrylpiperazin-1-yl)-2-(4-chlorophenylamino)ethanone;
1-(4-benzhydrylpiperazin-1-yl)-2-(3-chlorophenylamino)ethanone;
1-(4-benzhydrylpiperazin-1-yl)-2-(2-chlorophenylamino)ethanone;
1-(4-benzhydrylpiperazin-1-yl)-2-(2,4-difluorophenylamino)ethanone;
1-(4-benzhydrylpiperazin-1-yl)-2-(3,5-difluorophenylamino)ethanone;
1-(4-benzhydrylpiperazin-1-yl)-2-(3,5-dichlorophenylamino)ethanone;
1-(4-benzhydrylpiperazin-1-yl)-2-(3,5-dimethylphenylamino)ethanone;
1-(4-benzhydrylpiperazin-1-yl)-2-(4-fluorophenylamino)ethanone;
1-(4-benzhydrylpiperazin-1-yl)-2-(3,4,5-trimethoxyphenylamino)ethanone;
1-(4-benzhydrylpiperazin-1-yl)-2-(2,4-dichlorophenoxy)ethanone;
1-(4-benzhydrylpiperazin-1-yl)-2-(methyl(phenyl)amino)ethanone;
2-benzhydryl(methyl)amino-1-(4-(1-(1-methylpiperidin-4-yl)-1-phenylethyl)piperazin-1-yl)ethanone;
2-(benzhydrylamino)-1-(4-(bis(4-fluorophenyl)methyl)piperazin-1-yl)ethanone;
2-(benzhydryloxy)-1-(4-benzhydrylpiperazin-1-yl)ethanone;
2-(benzhydrylsulfinyl)-1-(2,6-dimethyl-4-((1-methylpiperidin-4-yl)methyl)piperazin-1-yl)ethanone;
2-(benzhydryloxy)-1-(2,6-dimethyl-4-((tetrahydro-2H-pyran-4-yl)methyl)piperazin-1-yl)ethanone;
2-(benzhydrylthio)-1-(2,6-dimethyl-4-((tetrahydro-2H-pyran-4-yl)methyl)piperazin-1-yl)ethanone;
2-(benzhydrylsulfinyl)-1-(2,6-dimethyl-4-((tetrahydro-2H-pyran-4-yl)methyl)piperazin-1-yl)ethanone;
2-(benzhydryloxy)-1-(4-((tetrahydro-2H-pyran-4-yl)methyl)piperazin-1-yl)ethanone;
2-(benzhydrylthio)-1-(4-((tetrahydro-2H-pyran-4-yl)methyl)piperazin-1-yl)ethanone;
2-(benzhydrylsulfinyl)-1-(4-((tetrahydro-2H-pyran-4-yl)methyl)piperazin-1-yl)ethanone;
2-(benzhydrylamino)-1-(4-((tetrahydro-2H-pyran-4-yl)methyl)piperazin-1-yl)ethanone;
2-(benzhydryloxy)-1-(4-((2-chloropyridin-4-yl)methyl)piperazin-1-yl)ethanone;
2-(benzhydryloxy)-1-(4-((2-chloro-6-methylpyridin-4-yl)methyl)piperazin-1-yl)ethanone;
2-(benzhydryloxy)-1-(4-((1-methylpiperidin-4-yl)methyl)piperazin-1-yl)ethanone;
2-(benzhydrylthio)-1-(4-((1-methylpiperidin-4-yl)methyl)piperazin-1-yl)ethanone;
2-(benzhydrylsulfinyl)-1-(4-((1-methylpiperidin-4-yl)methyl)piperazin-1-yl)ethanone;
1-(4-benzhydrylpiperazin-1-yl)-2-(benzhydrylthio)ethanone;
1-(4-benzhydrylpiperazin-1-yl)-2-(benzhydrylsulfinyl)ethanone;
2-(benzhydryloxy)-1-(4-(bis(4-fluorophenyl)methyl)piperazin-1-yl)ethanone;
2-(benzhydrylthio)-1-(4-(bis(4-fluorophenyl)methyl)piperazin-1-yl)ethanone;
2-(benzhydrylsulfinyl)-1-(4-(bis(4-fluorophenyl)methyl)piperazin-1-yl)ethanone;
2-benzhydrylamino-1-(4-benzhydryl-3-hydroxymethylpiperazin-1-yl)ethanone;
2-(benzhydryloxy)-1-(4-benzhydryl-3-hydroxymethylpiperazin-1-yl)ethanone;
1-(4-benzhydryl-3-hydroxymethylpiperazin-1-yl)-2-(benzhydrylsulfinyl)ethanone;
1-(4-benzhydryl-3-hydroxymethylpiperazin-1-yl)-2-(benzhydrylsulfinyl)ethanone;
2-benzhydrylamino-1-(4-benzhydryl-3-methoxymethylpiperazin-1-yl)ethanone;
2-(benzhydryloxy)-1-(4-benzhydryl-3-methoxymethyl piperazin-1-yl)ethanone;
1-(4-benzhydryl-3-methoxymethylpiperazin-1-yl)-2-(benzhydrylsulfinyl)ethanone;
1-(4-benzhydryl-3-methoxymethylpiperazin-1-yl)-2-(benzhydrylsulfinyl)ethanone;
2-benzhydrylamino-1-(4-benzhydryl-2-methoxymethylpiperazin-1-yl)ethanone;
and the pharmaceutically acceptable salts of any of these.

19. The method of claim 17 wherein the compound is:
2-benzhydrylamino-1-(4-benzhydrylpiperazin-1-yl)ethanone;
2-benzhydryl(methyl)amino-1-(4-benzhydrylpiperazin-1-yl)ethanone;
2-benzhydryl(methyl)amino-1-(4-((4-fluorophenyl)(phenyl)methyl)piperazin-1-yl)ethanone;
2-benzhydryl(methyl)amino-1-(4-((1-methylpiperidin-4-yl)(phenyl)methyl)piperazin-1-yl)ethanone;
1-(4-benzhydrylpiperazin-1-yl)-2-(2,4-dichlorophenoxy)ethanone;
2-(benzhydrylamino)-1-(4-(bis(4-fluorophenyl)methyl)piperazin-1-yl)ethanone;
2-(benzhydryloxy)-1-(4-benzhydrylpiperazin-1-yl)ethanone;
1-(4-benzhydrylpiperazin-1-yl)-2-(benzhydrylthio)ethanone;
1-(4-benzhydrylpiperazin-1-yl)-2-(benzhydrylsulfinyl)ethanone;
2-(benzhydryloxy)-1-(4-(bis(4-fluorophenyl)methyl)piperazin-1-yl)ethanone;
2-(benzhydrylsulfinyl)-1-(4-(bis(4-fluorophenyl)methyl)piperazin-1-yl)ethanone;
2-benzhydrylamino-1-(4-benzhydryl-3-hydroxymethylpiperazin-1-yl)ethanone;
or a pharmaceutically acceptable salt of one of these.

20. A compound of the formula: 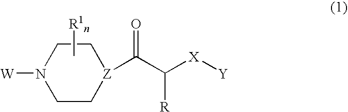
or a pharmaceutically acceptable salt or conjugates thereof,
wherein X is NR², O, S, S═O, or SO₂;
Z is N or CHNR²;
Y is CR³Ar₂ or Ar;
W is CR³Ar₂, CR³AB or C═OA,
wherein at least one of W and Y is CR³Ar₂;
each Ar is independently an optionally substituted aromatic or heteroaromatic ring;
A is an optionally substituted aromatic or heteroaromatic ring, or an optionally substituted carbocyclic or heterocyclic ring;
B is halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or B may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
R is H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R may be an optionally substituted group selected from alkyl (1-8C), alkenyl (2-8C), or alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
each R¹ is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10); or R¹ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
each R² is independently H, or an optionally substituted group selected from alkyl (1-8C), alkenyl (2-8C) and alkynyl (2-8C);
each R³ is independently H, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or R³ may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10C), heteroaryl (5-12C), and C6-C12-aryl-C1-C8-alkyl;
n is 0-4; and
one or more optional substituents may be on one or more of Ar, A or B wherein, when the substituents on Ar, A or B is on an aromatic or heteroaromatic group, each optional substituent is independently selected from halo, CN, NO₂, CF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10); or each optional substituent may be an optionally substituted group selected from alkyl (1-8C), alkenyl (2-8C), alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl; and
when the substituents on A or B is on a non-aromatic group, each substituent is independently selected from the group consisting of ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), heteroaryl (5-12C), and aryl (6-10); or each substituent may be independently selected from alkyl (1-8C), alkenyl (2-8C), or alkynyl (2-8C), heteroalkyl (2-8C), heteroalkenyl (2-8C), heteroalkynyl (2-8C), aryl (6-10), heteroaryl (5-12C), O-aryl (6-10), O-heteroaryl (5-12C) and C6-C12-aryl-C1-C8-alkyl;
with the proviso that if X is O and Y is phenyl, then Y is unsubstituted
and with the further proviso that if W is CR³AB and A is pyridine, then A is unsubstituted.

21. The compound of claim 20 wherein Z is N.

22. The compound of claim 20 wherein W is optionally substituted benzhydryl.

23. The compound of claim 20 wherein Y is optionally substituted benzhydryl.

24. The compound of claim 20, wherein W is CR³AB wherein A is 1-methylpiperidin-4-yl.

25. The compound of claim 24 wherein R³ is H.

26. The compound of claim 25 wherein B is H or optionally substituted phenyl.

27. The compound of claim 20 wherein Y is unsubstituted benzhydryl.

28. The compound of claim 20 wherein Y is optionally substituted phenyl.

29. The compound of claim 20 wherein X is NH or NMe.

30. The compound of claim 20 wherein each Ar is phenyl.

31. The compound of claim 20 wherein R is H.

32. The compound of claim 20 wherein n is 0 or 1 or 2.

33. The compound of claim 20 wherein the compound is selected from the group consisting of:
2-benzhydrylamino-1-(4-benzhydrylpiperazin-1-yl)ethanone;
2-benzhydryl(methyl)amino-1-(4-benzhydrylpiperazin-1-yl)ethanone;
2-benzhydryl(methyl)amino-1-(4-((4-chlorophenyl)(phenyl)methyl)piperazin-1-yl)ethanone;
2-benzhydryl(methyl)amino-1-(4-((3-chlorophenyl)(phenyl)methyl)piperazin-1-yl)ethanone;
2-benzhydryl(methyl)amino-1-(4-((2-chlorophenyl)(phenyl)methyl)piperazin-1-yl)ethanone;
2-benzhydryl(methyl)amino-1-(4-((4-fluorophenyl)(phenyl)methyl)piperazin-1-yl)ethanone;
2-benzhydryl(methyl)amino-1-(4-(phenyl(3-trifluoromethyl)phenyl)methyl)piperazin-1-yl)ethanone;
2-benzhydryl(methyl)amino-1-(4-(1-methyl-4-phenylpiperidine-4-carbonyl)piperazin-1-yl)ethanone;
2-benzhydrylamino-1-(4-((1-methylpiperidin-4-yl)piperazine-1-yl)ethanone;
2-benzhydrylamino-1-(4-(1-methylpiperidine-4-carbonyl)piperazin-1-yl)ethanone;
2-benzhydryl(methyl)amino-1-(4-(1-methylpiperidine-4-carbonyl)piperazin-1-yl)ethanone;
2-benzhydryl(methyl)amino-1-(4-((1-methylpiperidin-4-yl)piperazin-1-yl)ethanone;
(R)-2-(benzhydrylamino)-4-methyl-1-(4-((1-methylpiperidin-4-yl)methyl)piperazin-1-yl)pentan-1-one;
2-benzhydryl(methyl)amino-1-(4-((1-methylpiperidin-4-yl)(phenyl)methyl)piperazin-1-yl)ethanone;
2-benzhydryl(methyl)amino-1-(4-((4-fluorophenyl)(1-methylpiperidin-4-yl)methyl)piperazin-1-yl)ethanone;
1-(4-benzhydrylpiperazin-1-yl)-2-(phenylamino)ethanone;
1-(4-benzhydrylpiperazin-1-yl)-2-(4-chlorophenylamino)ethanone;
1-(4-benzhydrylpiperazin-1-yl)-2-(3-chlorophenylamino)ethanone;
1-(4-benzhydrylpiperazin-1-yl)-2-(2-chlorophenylamino)ethanone;
1-(4-benzhydrylpiperazin-1-yl)-2-(2,4-difluorophenylamino)ethanone;
1-(4-benzhydrylpiperazin-1-yl)-2-(3,5-difluorophenylamino)ethanone;
1-(4-benzhydrylpiperazin-1-yl)-2-(3,5-dichlorophenylamino)ethanone;
1-(4-benzhydrylpiperazin-1-yl)-2-(3,5-dimethylphenylamino)ethanone;
1-(4-benzhydrylpiperazin-1-yl)-2-(4-fluorophenylamino)ethanone;
1-(4-benzhydrylpiperazin-1-yl)-2-(3,4,5-trimethoxyphenylamino)ethanone;
1-(4-benzhydrylpiperazin-1-yl)-2-(2,4-dichlorophenoxy)ethanone;
1-(4-benzhydrylpiperazin-1-yl)-2-(methyl(phenyl)amino)ethanone;
2-benzhydryl(methyl)amino-1-(4-(1-(1-methylpiperidin-4-yl)-1-phenylethyl)piperazin-1-yl)ethanone;
2-(benzhydrylamino)-1-(4-(bis(4-fluorophenyl)methyl)piperazin-1-yl)ethanone;
2-(benzhydryloxy)-1-(4-benzhydrylpiperazin-1-yl)ethanone;
2-(benzhydrylsulfinyl)-1-(2,6-dimethyl-4-((1-methylpiperidin-4-yl)methyl)piperazin-1-yl)ethanone;
2-(benzhydryloxy)-1-(2,6-dimethyl-4-((tetrahydro-2H-pyran-4-yl)methyl)piperazin-1-yl)ethanone;
2-(benzhydrylthio)-1-(2,6-dimethyl-4-((tetrahydro-2H-pyran-4-yl)methyl)piperazin-1-yl)ethanone;
2-(benzhydrylsulfinyl)-1-(2,6-dimethyl-4-((tetrahydro-2H-pyran-4-yl)methyl)piperazin-1-yl)ethanone;
2-(benzhydryloxy)-1-(4-((tetrahydro-2H-pyran-4-yl)methyl)piperazin-1-yl)ethanone;
2-(benzhydrylthio)-1-(4-((tetrahydro-2H-pyran-4-yl)methyl)piperazin-1-yl)ethanone;
2-(benzhydrylsulfinyl)-1-(4-((tetrahydro-2H-pyran-4-yl)methyl)piperazin-1-yl)ethanone;
2-(benzhydrylamino)-1-(4-((tetrahydro-2H-pyran-4-yl)methyl)piperazin-1-yl)ethanone;
2-(benzhydryloxy)-1-(4-((2-chloropyridin-4-yl)methyl)piperazin-1-yl)ethanone;
2-(benzhydryloxy)-1-(4-((2-chloro-6-methylpyridin-4-yl)methyl)piperazin-1-yl)ethanone;
2-(benzhydryloxy)-1-(4-((1-methylpiperidin-4-yl)methyl)piperazin-1-yl)ethanone;
2-(benzhydrylthio)-1-(4-((1-methylpiperidin-4-yl)methyl)piperazin-1-yl)ethanone;
2-(benzhydrylsulfinyl)-1-(4-((1-methylpiperidin-4-yl)methyl)piperazin-1-yl)ethanone;
1-(4-benzhydrylpiperazin-1-yl)-2-(benzhydrylthio)ethanone;
1-(4-benzhydrylpiperazin-1-yl)-2-(benzhydrylsulfinyl)ethanone;
2-(benzhydryloxy)-1-(4-(bis(4-fluorophenyl)methyl)piperazin-1-yl)ethanone;
2-(benzhydrylthio)-1-(4-(bis(4-fluorophenyl)methyl)piperazin-1-yl)ethanone;
2-(benzhydrylsulfinyl)-1-(4-(bis(4-fluorophenyl)methyl)piperazin-1-yl)ethanone;
2-benzhydrylamino-1-(4-benzhydryl-3-hydroxymethylpiperazin-1-yl)ethanone;
2-(benzhydryloxy)-1-(4-benzhydryl-3-hydroxymethylpiperazin-1-yl)ethanone;
1-(4-benzhydryl-3-hydroxymethylpiperazin-1-yl)-2-(benzhydrylsulfinyl)ethanone;
1-(4-benzhydryl-3-hydroxymethylpiperazin-1-yl)-2-(benzhydrylsulfinyl)ethanone;
2-benzhydrylamino-1-(4-benzhydryl-3-methoxymethylpiperazin-1-yl)ethanone;
2-(benzhydryloxy)-1-(4-benzhydryl-3-methoxymethyl piperazin-1-yl)ethanone;
1-(4-benzhydryl-3-methoxymethylpiperazin-1-yl)-2-(benzhydrylsulfinyl)ethanone;
1-(4-benzhydryl-3-methoxymethylpiperazin-1-yl)-2-(benzhydrylsulfinyl)ethanone;
2-benzhydrylamino-1-(4-benzhydryl-2-methoxymethylpiperazin-1-yl)ethanone; and
the pharmaceutically acceptable salts of any of these.

34. The compound of claim 33 wherein the compound is:
2-benzhydrylamino-1-(4-benzhydrylpiperazin-1-yl)ethanone;
2-benzhydryl(methyl)amino-1-(4-benzhydrylpiperazin-1-yl)ethanone;
2-benzhydryl(methyl)amino-1-(4-((4-fluorophenyl)(phenyl)methyl)piperazin-1-yl)ethanone;
2-benzhydryl(methyl)amino-1-(4-((1-methylpiperidin-4-yl)(phenyl)methyl)piperazin-1-yl)ethanone;
1-(4-benzhydrylpiperazin-1-yl)-2-(2,4-dichlorophenoxy)ethanone;
2-(benzhydrylamino)-1-(4-(bis(4-fluorophenyl)methyl)piperazin-1-yl)ethanone;
2-(benzhydryloxy)-1-(4-benzhydrylpiperazin-1-yl)ethanone;
1-(4-benzhydrylpiperazin-1-yl)-2-(benzhydrylthio)ethanone;
1-(4-benzhydrylpiperazin-1-yl)-2-(benzhydrylsulfinyl)ethanone;
2-(benzhydryloxy)-1-(4-(bis(4-fluorophenyl)methyl)piperazin-1-yl)ethanone;
2-(benzhydrylsulfinyl)-1-(4-(bis(4-fluorophenyl)methyl)piperazin-1-yl)ethanone;
2-benzhydrylamino-1-(4-benzhydryl-3-hydroxymethylpiperazin-1-yl)ethanone; and
a pharmaceutically acceptable salt of one of these.

35. A pharmaceutical composition which comprises the compound of claim 20 in admixture with a pharmaceutically acceptable excipient.

36-39. (canceled)
